(19) United States
(12) Patent Application Publication
(10) Pub. No.: US 2025/0204515 A1
(43) Pub. Date: Jun. 26, 2025

(54) Heparanase Inhibition for Graft Protection

(71) Applicant: University of Pittsburgh - Of the Commonwealth System of Higher Education, Pittsburgh, PA (US)

(72) Inventors: Pablo Gerardo Sanchez, Pittsburgh, PA (US); Kentaro Noda, Pittsburgh, PA (US)

(21) Appl. No.: 18/847,735

(22) PCT Filed: Mar. 17, 2023
(86) PCT No.: PCT/IB2023/052643

§ 371 Date: Sep. 17, 2024

Related U.S. Application Data
(60) Provisional application No. 63/321,370, filed on Mar. 18, 2022.

(51) Int. Cl. A01N 1/126; A61K 31/445; A61K 45/06; A61P 37/06; C12N 9/99

(52) U.S. Cl. CPC A01N 1/126; A61K 31/445; A61K 45/06; A61P 37/06; C12N 9/99

(57) ABSTRACT

Methods are provided for preparing and treating graft tissue, such as a solid organ, for example a lung, comprising contacting the graft tissue with protective amounts of a heparanase inhibitor prior to and/or after transplantation of the graft tissue. Graft tissue, such as lung tissue prepared by the method also are provided. Methods are provided for transplanting graft tissue, such as a solid organ, for example a lung, comprising contacting the graft tissue with protective amounts of a heparanase inhibitor prior to and/or after transplantation of the graft tissue.

### CROSS REFERENCE TO RELATED APPLICATIONS

This application is the United States national phase of International Patent Application No. PCT/IB2023/052643 filed Mar. 17, 2023, and claims priority to U.S. Provisional Patent Application No. 63/321,370 filed Mar. 18, 2022, the disclosures of which are hereby incorporated by reference in their entireties.

### BACKGROUND OF THE INVENTION

Solid-organ transplantation is the only therapeutic option for patients with severe end stage organ failure/diseases. In general, graft protection is a matter to address for donor shortage, graft preservation, and graft rejection after transplantation. Post-transplant patient death occurs in a broad timeline with graft failure. Therapies improving or repairing the quality of donated tissues and organs before transplant contribute to improving outcomes. Endothelial cells are the primary cell type that contacts blood and are responsible for regulating vascular homeostasis, maintaining the fluid barrier, controlling cellular adhesion and immune cell extravasation. These functions are directly associated with graft function, quality, and rejection, thus, endothelial protection is a key factor to protect grafts and improve post-transplant outcomes. The endothelial glycocalyx is a protective layer covering the endothelial surface. Upon the damage to the endothelium, the endothelial glycocalyx is dissociated by responsible enzymes, leading to further endothelial dysfunction.

Methods and compositions for improving graft outcomes are needed.

### SUMMARY

Provided herein is method of preparing graft tissue for transplantation, comprising contacting the tissue with an amount of a heparanase inhibitor effective to reduce transplant rejection of the tissue in a recipient patient in which the tissue is transplanted.

Also provided herein is method for transplanting graft tissue from a donor patient to a recipient patient, comprising, implanting the tissue into the recipient patient, and prior to or after transplanting the tissue into the recipient, contacting the tissue with an amount of a heparanase inhibitor compound effective to reduce transplant rejection of the tissue in a recipient patient in which the tissue is transplanted.

The following numbered clauses outline various exemplary embodiments and aspects of the present invention.

Clause 1. A method of preparing graft tissue for transplantation, comprising contacting the tissue with an amount of a heparanase inhibitor effective to reduce transplant rejection of the tissue in a recipient patient in which the tissue is transplanted.

Clause 2. The method of clause 1, wherein the heparanase inhibitor is selected from the group consisting of: heparin, chemical derivatives of heparin, nonanticoagulant heparin, sulfated phosphomannopentaose (PI-88, Mupafostat), sulfated tri mannose C—C-linked dimers, trachyspic acid, trachyspic acid 19-butyl ester, oligomannurarate sulfate (JG3), SST0001 (Roneparstat), M402 (Necuparanib), laminaran sulfate, PG545 (Pixatimod) and its analogs, 2-[4-propylamino-5-[5-(4-chloro)phenyl-benzoxazol-2-yl]phenyl]-2,3-dihydro-1,3-dioxo-1 H-isoindole-5-carboxylic acid, benzoxazol-5-ylacetic acids, RK-682 (3-hexadecanoyl-5-hydroxymethyltetronic acid), 1-[4-H-benzoimidazol-2-yl]-phenyl]-3-[4-(1 H-benzoimidazol-2-yl)-phenyl]-ureas such as 1,3-bis-[4-(1 H-benzoimidazol-2-yl)-phenyl]-urea, RK-682 series compounds such as 4-Bn-RK-682, KI-105 series compounds, heparin and heparin sulfate-binding fragments of heparanase, defibrotide, RG-13577, and an antiheparanase antibody, or a pharmacologically acceptable salt or isostere of any of the preceding.

Clause 3. The method of clause 1, the compound having the structure:

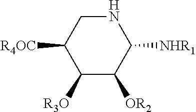

wherein:
R₁ is a hydrogen atom, an unsubstituted or halogen-substituted, straight or branched acyl group of 1-5 carbon atoms (C₁₋₅ acyl), or —C(═NH)R₅ in which R₅ is a group —OR₆ or —NR₇R₈ where R₆, R₇, and R₈ each stand for a hydrogen atom or a straight or branched alkyl group of 1-5 carbon atoms (C₁₋₅ alkyl);
R₂ and R₃ may be the same or different from each other and are a hydrogen atom or an unsubstituted or halogen-substituted, straight or branched C₁₋₅ acyl; and R₄ is —OR₉ or —NR₁₀R₁₁ where R₉, R₁₀, and R₁₁ each stand for, independently, a hydrogen atom or a straight or branched C₁₋₅ alkyl group, or a pharmacologically acceptable salt or isostere thereof.

Clause 4. The method of clause 3, wherein R₁, R₂, and/or R₃ are, independently a C₁₋₅ alkanoyl group.

Clause 5. The method of clause 1, wherein the heparanase inhibitor compound comprises heparastatin, or a pharmaceutically-acceptable salt or isostere thereof, e.g., heparastatin HCl having the exemplary structure:

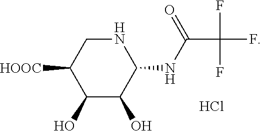 [SMILES: O=C(O)[C@H]1CN[C@H](NC(=O)C(F)(F)F)[C@@H](O)[C@H]1O ; Cl]

Clause 6. The method of any one of clauses 1-5, wherein the tissue is contacted with the compound while the tissue is in a donor of the tissue.

Clause 7. The method of any one of clauses 1-6, wherein the tissue is contacted with the compound ex vivo or in vitro.

Clause 8. The method of any one of clauses 1-7, wherein the tissue is contacted with the compound after implantation in a recipient of the tissue.

Clause 9. The method of any one of clauses 1-8, wherein the tissue is in the form of an organ.

Clause 10. The method of clause 9, wherein the organ is perfused with a perfusate comprising the heparanase inhibitor compound.

Clause 11. The method of any one of clauses 1-10, wherein the tissue is contacted with a solution of the heparanase inhibitor compound in which the concentration of the heparanase inhibitor compound ranges from 10 nM (nanomolar) to 5 mM (millimolar).

Clause 12. The method of any one of clauses 5-10, wherein the tissue is contacted with a solution of the heparastatin in which the concentration of the heparastatin ranges from 10 nM to 5 mM.

Clause 13. The method of clause 11 or 12, wherein the tissue is contacted with the compound in a single bolus, or in multiple doses, intermittently, or continuously over a time period ranging from 10 minutes, 20 minutes, 30 minutes, 45 minutes, 1 hour, 2 hours, 3 hours, 4 hours, 5 hours, 6 hours, 12 hours, or 24 hours, or any increment therebetween.

Clause 14. The method of any one of clauses 1-13, wherein the tissue comprises lung tissue, e.g. the tissue is a lung.

Clause 15. The method of any one of clauses 5-14, wherein the heparanase is heparastatin, which is contacted with the tissue at a concentration of at least 10 nMO nM, at least 50 nM, at least 100 nM, at least 500 nM, at least 1 μM, at least 10 μM, at least 25 μM, or at least 50 μM and, optionally, no greater than 5 mM.

Clause 16. The method of any one of clauses 1-14, wherein the heparanase inhibitor is contacted with the tissue at a therapeutic equivalent concentration to a concentration of SF-4 (heparastatin) of at least 10 nM, at least 50 nM, at least 100 nM, at least 500 nM, at least 1 μM, at least 10 μM, at least 25 μM, or at least 50 μM and, optionally, no greater than 5 mM.

Clause 17. Graft tissue, such as lung tissue or a lung, prepared according to a method of any one of clauses 1-16.

Clause 18. A method for transplanting graft tissue from a donor patient to a recipient patient, comprising, implanting the tissue into the recipient patient, and prior to or after transplanting the tissue into the recipient, contacting the tissue with an amount of a heparanase inhibitor compound effective to reduce transplant rejection of the tissue in a recipient patient in which the tissue is transplanted.

Clause 19. The method of clause 18, wherein the heparanase inhibitor is selected from the group consisting of: heparin, chemical derivatives of heparin, nonanticoagulant heparin, sulfated phosphomannopentaose (PI-88, Mupafostat), sulfated tri mannose C—C-linked dimers, trachyspic acid, trachyspic acid 19-butyl ester, oligomannurarate sulfate (JG3), SST0001 (Roneparstat), M402 (Necuparanib), laminaran sulfate, PG545 (Pixatimod) and its analogs, 2-[4-propylamino-5-[5-(4-chloro)phenyl-benzoxazol-2-yl]phenyl]-2,3-dihydro-1,3-dioxo-1 H-isoindole-5-carboxylic acid, benzoxazol-5-ylacetic acids, RK-682 (3-hexadecanoyl-5-hydroxymethyltetronic acid), 1-[4-H-benzoimidazol-2-yl]-phenyl]-3-[4-(1 H-benzoimidazol-2-yl)-phenyl]-ureas such as 1,3-bis-[4-(1 H-benzoimidazol-2-yl)-phenyl]-urea, RK-682 series compounds such as 4-Bn-RK-682, KI-105 series compounds, heparin and heparin sulfate-binding fragments of heparanase, defibrotide, RG-13577, and an antiheparanase antibody, or a pharmacologically acceptable salt or isostere of any of the preceding.

Clause 20. The method of clause 18, the compound having the structure:

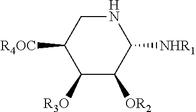

wherein:
R₁ is a hydrogen atom, an unsubstituted or halogen-substituted, straight or branched acyl group of 1-5 carbon atoms (C₁₋₅ acyl), or —C(═NH)R₅ in which R₅ is a group —OR₆ or —NR₇R₈ where R₆, R₇, and R₈ each stand for a hydrogen atom or a straight or branched alkyl group of 1-5 carbon atoms (C₁₋₅ alkyl); R₂ and R₃ may be the same or different from each other and are a hydrogen atom or an unsubstituted or halogen-substituted, straight or branched C₁₋₅ acyl; and R₄ is —OR₉ or —NR₁₀R₁₁ where R₉, R₁₀, and R₁₁ each stand for, independently, a hydrogen atom or a straight or branched C₁₋₅ alkyl group, or a pharmacologically acceptable salt or isostere thereof.

Clause 21. The method of clause 20, wherein R₁, R₂, and/or R₃ are, independently a C₁₋₅ alkanoyl group.

Clause 22. The method of clause 18, wherein the heparanase inhibitor compound comprises heparastatin, or a pharmaceutically-acceptable salt or isostere thereof, e.g., heparastatin HCl having the exemplary structure:

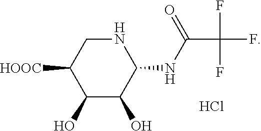 [SMILES: O=C(O)[C@H]1CN[C@H](NC(=O)C(F)(F)F)[C@@H](O)[C@H]1O ; Cl]

Clause 23. The method of any one of clauses 18-22, wherein the tissue is contacted with the compound while the tissue is in a donor of the tissue.

Clause 24. The method of any one of clauses 18-23, wherein the tissue is contacted with the compound ex vivo or in vitro.

Clause 25. The method of any one of clauses 18-24, wherein the tissue is contacted with the compound after implantation in a recipient of the tissue.

Clause 26. The method of any one of clauses 18-25, wherein the tissue is in the form of an organ.

Clause 27. The method of clause 26, wherein the organ is perfused with a perfusate comprising the heparanase inhibitor compound.

Clause 28. The method of any one of clauses 18-27, wherein the tissue is contacted with a solution of the heparanase inhibitor compound in which the concentration of the heparanase inhibitor compound ranges from 10 nM to 5 mM.

Clause 29. The method of any one of clauses 18-27, wherein the tissue is contacted with a solution of the heparastatin in which the concentration of the heparastatin ranges from 10 nM to 5 mM.

Clause 30. The method of clause 28 or 29, wherein the tissue is contacted with the compound in a single bolus, or in multiple doses, intermittently, or continuously over a time period ranging from 10 minutes, 20 minutes, 30 minutes, 45 minutes, 1 hour, 2 hours, 3 hours, 4 hours, 5 hours, 6 hours, 12 hours, or 24 hours, or any increment therebetween.

Clause 31. The method of any one of clauses 18-30, wherein the tissue comprises lung tissue, e.g. the tissue is a lung.

Clause 32. The method of any one of clauses 18-31, wherein the heparanase inhibitor is heparastatin, which is contacted with the tissue at a concentration of at least 10 nM, at least 50 nM, at least 100 nM, at least 500 nM, at least 1 μM, at least 10 μM, at least 25 μM, or at least 50 μM, and, optionally, no greater than 5 mM.

Clause 33. The method of any one of clauses 18-31, wherein the heparanase inhibitor is contacted with the tissue at a therapeutic equivalent concentration to a concentration of SF-4 (heparastatin) of at least 10 nM, at least 50 nM, at least 100 nM, at least 500 nM, at least 1 μM, at least 10 μM, at least 25 μM, or at least 50 μM, and, optionally, no greater than 5 mM.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. (a) Representative images of rat lungs after procurement. (b) Heparanase activity was detected in tissue after procurement following 1 h of warm ischemia. (c) Ultrastructural images of the endothelial glycocalyx (eGC) from lung grafts after 1 h of warm ischemia obtained using transmission electron microscopy. The labeled glycocalyx appears as the darker cell surface layer; black arrow indicates glycocalyx desquamated from the surface of an endothelial cell. *p<0.05, **p<0.01. NL native lungs, Ctrl control lungs with eGC damage induced by 1 h of ischemia, Hep lungs with eGC preserved by heparin administration prior to 1 h of ischemia, NAH lungs with intact eGC preserved by N-acetyl heparin administration prior to 1 h of ischemia.

FIG. 2. The effect of eGC damage in the lung grafts on the post-transplant graft function and quality. Grafts were evaluated 2 h after transplantation for (a) PaO2/FiO2 (P/F ratio), (b) macroscopic morphology (representative samples are shown), (c) Wet-to-dry (W/D) ratio, and (d) microscopic morphology (representative H&E-stained samples are shown). *p<0.05, **p<0.01. NL native lungs, Ctrl control lungs with eGC damage prior to transplant, Heparin lungs with eGC preserved by heparin prior to transplant, NAH lungs with eGC preserved by N-acetyl heparin prior to transplant, h/R injury ischemia reperfusion injury.

FIG. 3. Endothelial glycocalyx influences the inflammation and inflammatory cell migration in lung grafts after transplantation. (a) Real-time RT-PCR for the mRNA of pro-inflammatory cytokines interleukin (IL)-6 and IL-1p. (b) The extravasation of neutrophils, T cells, and monocytes in the transplanted grafts were evaluated by specific staining of polymorphonuclear neutrophils (naphthol staining, positive cells indicated by black arrow heads), T cells (CD3+-positive, indicated by white arrows) and macrophages (CD68+-positive, purple staining), and (c) quantitated. *p<0.05, **p<0.01. NL native lungs, Ctrl control lungs with eGC damage prior to transplant, Heparin lungs with eGC preserved by heparin prior to transplant, NAH lungs with eGC preserved by N-acetyl heparin prior to transplant.

FIG. 4. Images of full length of western blotting for vascular endothelial (VE)-cadherin, heat shock protein 90 (HSP90), and syndecan-1 to examine membrane and cytosol proteins in all fractionated lung tissue samples collected after transplantation. Membranes were routinely cut between the 75 kDa and 50 kDa molecular weight markers to assess expression of multiple proteins in the same sample. The images for membrane proteins and cytosol proteins were obtained under the same conditions (e.g. antibody incubation, antibody concentration, blocking time and exposure time) for each blot. Mk, molecular weight marker. NL, native lungs; Control, control lungs with the endothelial glycocalyx (eGC) damage prior to transplant; Heparin, lungs with eGC preserved by heparin prior to transplant; NAH, lungs with eGC preserved by N-acetyl heparin prior to transplant.

FIG. 5. Assessment of endothelial glycocalyx and microvascular integrity in lung grafts after reperfusion. (a) Western blot of syendecan-1 in fractionated lung tissue 2 h after transplantation. Gel image depicts a single representative sample from each treatment group. Vascular endothelial cadherin (VE-Cad) and heat shock protein 90 (HSP90) were blotted as loading markers for the plasma membrane and cytosolic protein fractions, respectively. Cytosol protein fraction, M membrane protein fraction. (b) Glycosaminoglycan (GAG) content in the grafts 2 h after transplantation. (c) Syndecan-1 staining (yellow) in the peripheral vasculature (φ˜50 mm) of the grafts 2 h after transplantation. The endothelial luminal surface is indicated as a white line in the merged images (right column). Nuclei were stained using Hoechst 33342. BAF background autofluorescence, RBCs red blood cells, VL vascular lumen. (d) Pulmonary vascular resistance (PVR) of lung grafts during ex vivo lung perfusion (EVLP). Time-dependent fold changes from the values at 1 h are shown. n=4-5 for each group. (e) Endothelial barrier integrity after EVLP was assessed by measuring the amount of Evans blue dye (EBD) retained in the tissue. (f) Time-dependent changes of syndecan-1 in the perfusate during EVLP. *p<0.05, **p<0.01. NL native lungs, Ctrl control lungs with eGC damage prior to transplant/EVLP, Hep lungs with eGC preserved by heparin treatment prior to transplant or EVLP, NAH lungs with eGC preserved by N-acetyl heparin treatment prior to transplant or EVLP.

FIG. 6. Representative images of immunofluorescent staining for syndecan-1 in the peripheral airways in lung grafts 2 hours after transplantation and reperfusion. The region where the respiratory bronchioles (RB) meet the alveolar ducts (AD) was defined as the peripheral airway. Syndecan-1 is visualized in yellow, and nuclei are stained with Hoechst 33342 (shown as blue), and the entire microscopic structure was visualized using background autofluorescence (BAF). Control, experimental model of lung grafts with damaged endothelial glycocalyx (eGC) prior to transplant; Heparin, experimental model of lung grafts with eGC preserved by heparin administration prior to transplant; NAH, experimental model of lungs with eGC preserved by N-acetyl heparin administration prior to transplant.

FIG. 7. The effects of heparanase inhibition on MMP-2 and MMP-9 activity. (a) The mRNA expression of metalloproteinase (MMP)-9 in lung grafts 2 h after transplantation. (b) Representative gelatin zymography of time-dependent changes in activity of secreted MMP-2 and MMP-9 in EVLP perfusate. (c) MMP-2 activity quantitated and shown as fold change. *p<0.05, **p<0.01. NL native lungs, Ctrl control, Heparin, NAH N-acetyl heparin.

FIG. 8. Dose responses of in vitro β-D-glucuronidase activity against the heparanase inhibitor candidates. The β-D-glucuronidase activity was altered in the presence of (A) heparastatin [SF4], (B) heparin, (C) amodiaquine, (D) triazolothiadiasoles, and (E) OGT-2113 (structure below), in a dose-dependent manner.

FIG. 9. Clinical correlation of glycocalyx degradation with graft quality during ex vivo lung perfusion (EVLP) and primary graft dysfunction (PGD). (A,B) The temporal change of concentrations of (A) heparan sulfate and (B) syndecan-1 in perfusate between 1 h and the end of EVLP. Data were analyzed by two-way ANOVA with repeated measures followed by post hoc Tukey HSD test. *p=0.016 between PGD3 and declined lungs in heparan sulfate. No other significant differences were determined among groups in (A) heparan sulfate and (B) syndecan-1. (C,D) The shedding rate of major glycocalyx components (C) heparan sulfate and (D) syndecan-1 in lung grafts from brain death donors during assessment on EVLP and the decision to transplant. The data shows lung grafts deemed not suitable for transplantation on EVLP (Declined, n=7) and lung grafts transplanted after EVLP (Transplanted, n=17). iBW: ideal body weight. *p=0.034 were determined by Mann-Whitney U test. (E,F) Clinical predictive value of (E) heparan sulfate and (F) syndecan-1 concentration in perfusate at the end of EVLP was compared to the incidence of severe PGD at posttransplant 72 hours. *p=0.015 were determined by Mann-Whitney U test.

FIG. 10. Physiologic parameters and endothellal damage In rat lungs during EVLP. (A-C) Lung physiology was evaluated by (A) PaO2/FiO2 (P/F) ratio (B) pulmonary vascular resistance (PVR) and (C) lung compliance (Cdyn) during EVLP. n=16 in control, 13 in heparin and 9 in SF4. Data were analyzed by two-way ANOVA with repeated measures followed by post hoc Tukey HSD test. (D) Representative gross visual images of lungs after Evans blue dye (EBD) perfusion to assess endothelial barrier function in the lungs after 4 hours of EVLP. (E) Quantitation of EBD in lung tissue. **p<0.01 determined by one-way ANOVA followed by post hoc analysis with the Bonferroni correction. (F) Wet-to-dry ratio of lung tissue after 4 hours of EVLP. *p<0.05, **p<0.01 determined by one-way ANOVA followed by post hoc analysis with the Bonferroni correction. Control: EVLP with no treatment, Heparin: EVLP with heparin administration, SF4: EVLP with heparastatin (SF4) administration. Wt, weight.

FIG. 11. Expression and activity of glycocalyx-dissociation enzymes in rat lungs during EVLP. (A) The activity of heparanase (HPSE) in rat lungs after 4 hours of EVLP. **p<0.01 determined by one-way ANOVA followed by post hoc analysis with the Bonferroni correction. (B) Representative image of gelatin zymography to see the activity of matrix metalloproteinase (MMP)-2 and MMP-9 secreted into the perfusate during 4 hours of EVLP. Multiple samples for each treatment were loaded on each gel. (C) Fold change in MMP-2 activity as compared with controls. **p<0.01 determined by one-way ANOVA followed by post hoc analysis with the Bonferroni correction. (D,E) Expression of (D) heparanase-1 and (E) MMP-2 mRNAs in lung tissue after 4 hours of EVLP. *p<0.05, **p<0.01 determined by one-way ANOVA followed by post hoc analysis with the Bonferroni correction. (F,G) Dissociated glycocalyx components as shown (F) glycosaminoglycans (GAGs) and (G) Syndecan-1 protein concentration in EVLP perfusate at 4 hours EVLP. **p<0.01 determined by one-way ANOVA followed by post hoc analysis with the Bonferroni correction. Control, EVLP with no treatment; Heparin (Hep), EVLP with heparin administration; SF4, EVLP with heparastatin (SF4) administration.

FIG. 12. Syndecan-1 shedding in rat lungs during EVLP. Representative images of immunofluorescent staining for syndecan-1 (red), caveolin-1 (white) and nuclei (blue) in rat lungs after 4 hours of EVLP. Syndecan-1 is an integral component of the glycocalyx; caveolin-1 is an endothelial cell marker. Hoechst 33342 dye was used for nuclear staining. White bar=100 μm. EVLP control, EVLP with no treatment; EVLP heparin, EVLP with heparin administration; EVLP SF4, EVLP with heparastatin (SF4) administration. Cav-1, caveolin-1; Synd-1, syndecan-1.

FIG. 13. Assessment of proinflammatory pathway activation in rat lungs after EVLP. (A-C) Real-time RT-PCR was performed to quantitate the mRNA expression levels for proinflammatory cytokines in rat lung tissue after 4 hours of EVLP. (A) interleukin (IL)-6, (B) IL-1β, and (C) TNF-α. *p=0.05, **p<0.01 determined by one-way ANOVA followed by post hoc analysis with the Bonferroni correction. (D) Nuclear factor (NF)-κB activation was examined in lung tissue after 4 hours of EVLP by the western blotting for inhibitor of KB-α (IκB-α). β-actin levels were used as an internal control. Relative intensity of IκB-α is shown by a comparison to β-actin intensity and normalized by the first left line of controls (set as 1.00). *p<0.05, determined by one-way ANOVA followed by post hoc analysis with the Bonferroni correction. Control (Ctrl), EVLP with no treatment; Heparin (Hep), EVLP with heparin administration; SF4, EVLP with heparastatin (SF4) administration.

FIG. 14. Assessment of lung grafts after EVLP and transplantation. (A) Lung function (PaO2/FiO2) in grafts 2 hours after transplant following 4 hours of EVLP. **p<0.01 determined by one-way ANOVA followed by post hoc analysis with the Bonferroni correction. (B) Representative images of H&E-stained lung grafts 2 hours after transplant following 4 hours of EVLP. Scale bar=100 μm. (C) Real-time RT-PCR for pro-inflammatory mediators (IL-6, IL-13 and TNF-α) in lung grafts 2 hours after transplant following 4 hours of EVLP. *p<0.05, **p<0.01 determined by one-way ANOVA followed by post hoc analysis with the Bonferroni correction. Control (Ctrl), EVLP with no treatment; Heparin (Hep), EVLP with heparin administration; SF4, EVLP with heparastatin (SF4) administration.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention. The term “consisting of” excludes any element, step, or ingredient not specified in the claim. As used herein, embodiments “comprising” one or more stated elements or steps also include, but are not limited to embodiments “consisting essentially of” and “consisting of” these stated elements or steps.

As used herein, the “treatment” or “treating” of a patient means administration to a patient by any suitable dosage regimen, procedure and/or administration route of a composition, device, or structure with the object of achieving a desirable clinical/medical end-point, including but not limited to, increased survival, reduction of inflammation, reduction of graft deterioration, degeneration, or rejection, and/or improvement of any other suitable symptom or marker of graft deterioration or rejection. An amount of any reagent or therapeutic agent, administered by any suitable route, effective to treat a patient is an amount capable of reducing graft deterioration, degeneration, or rejection in a graft recipient. The therapeutically-effective amount of each therapeutic may range from 1 μg per dose to 10 g per dose, including any amount there between, such as, without limitation, 1 ng, 1 μg, 1 mg, 10 mg, 100 mg, 1 g, or 10 g per dose, where a dose may be an amount administered to a patient, or perfused into an organ either in vivo or ex vivo., in a pharmaceutically-acceptable carrier For example, a dose may provide an effective concentration of the heparanase inhibitor, such as heparastatin, to a patient, or to cells, tissue, an organ or a graft, such as, without limitation, 10 nM to 5 mM. In the case of heparastatin, the compound may be administered or contacted with tissue at a concentration of at least 10 nM, at least 50 nM, at least 100 nM, at least 500 nM, at least 1 μM, at least 10 μM, at least 25 μM, or at least 50 μM, and, optionally, no greater than 5 mM. In the case of other heparanase inhibitors, the heparanase inhibitor may be contacted with the tissue at a therapeutic equivalent concentration to a concentration of SF-4 (heparastatin) of at least 10 nM, at least 50 nM, at least 100 nM, at least 500 nM, at least 1 μM, at least 10 μM, at least 25 μM, or at least 50 μM, and, optionally, no greater than 5 mM.

The therapeutic agent may be administered by any effective route, but in the context of prevention, treatment, or reduction of graft deterioration, degeneration, or rejection, may be most typically delivered parenterally to a donor or recipient of a graft, or by in vivo or ex vivo perfusion of an organ or tissue. In vivo perfusion may be performed prior to removal of a graft material from a donor, or after implantation of a graft in a recipient. The therapeutic agent may be administered as a single dose, at regular or irregular intervals, in amounts and intervals as dictated by any clinical parameter of a patient or graft organ or tissue, or continuously.

Active ingredients, such as the compounds described herein, may be compounded or otherwise manufactured into a suitable composition for use, such as a pharmaceutical dosage form or drug product in which the compound is an active ingredient. Compositions may comprise a pharmaceutically acceptable carrier, or excipient. An excipient is an inactive substance used as a carrier for the active ingredients of a medication. Although “inactive,” excipients may facilitate and aid in increasing the delivery or bioavailability of an active ingredient in a drug product. Non-limiting examples of useful excipients include: anti-adherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts. A compound may be delivered in a lipid nanoparticle.

Useful dosage forms include: intravenous, perfusate, intramuscular, intraocular, or intraperitoneal solutions, oral tablets or liquids, topical ointments or creams, and transdermal devices (e.g., patches). The compound may be an intravenous liquid or emulsion or a perfusate liquid or emulsion.

Suitable dosage forms may include single-dose, or multiple-dose vials or other containers, such as medical syringes or IV bags, containing a composition comprising an active ingredient useful for the methods described herein.

Pharmaceutical formulations adapted for administration include aqueous and non-aqueous sterile solutions which may contain, for example and without limitation, anti-oxidants, buffers, bacteriostats, lipids, liposomes, lipid nanoparticles, emulsifiers, suspending agents, and rheology modifiers. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous solutions and suspensions may be prepared from sterile powders, granules, and tablets. Therapeutic compositions typically must be sterile and stable under the conditions of manufacture and storage. For example, sterile injectable solutions can be prepared by incorporating the active agent in the required amount in an appropriate solvent with one or a combination of ingredients enumerated herein, as required, followed by filtered sterilization. Generally, dispersions are prepared by incorporating the active compound into a sterile vehicle that contains a basic dispersion medium and any required or otherwise selected other ingredients. In the case of sterile powders for the preparation of sterile injectable solutions, typical methods of preparation are vacuum drying and freeze-drying that yields a powder of the active ingredient plus any additional desired ingredient from a previously sterile-filtered solution thereof.

The phrase “pharmaceutically-acceptable carrier” as used herein can refer to a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier can be “acceptable” in the sense of being compatible with the other ingredients of the formulation and not substantially injurious to the subject being treated. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (24) other non-toxic compatible substances employed in pharmaceutical formulations.

A “therapeutically effective amount” refers to an amount of a drug product or active agent effective, at dosages and for periods of time necessary, to achieve the desired therapeutic result. An “amount effective” for treatment of a condition is an amount of an active agent or dosage form, such as a single dose or multiple doses, effective to achieve a determinable end-point. The “amount effective” is preferably safe—at least to the extent the benefits of treatment outweighs the detriments, and/or the detriments are acceptable to one of ordinary skill and/or to an appropriate regulatory agency, such as the U.S. Food and Drug Administration. A therapeutically effective amount of an active agent, in the case of a perfusate for reduction or prevention of graft deterioration, degeneration, or rejection (collectively, graft protection) may vary according to factors such as the selected organ and the ability of the active agent to elicit a desired response in the individual. A “prophylactically effective amount” refers to an amount effective, at dosages and for periods of time necessary, to achieve a desired prophylactic result, (e.g., for graft protection.)

Dosage regimens may be adjusted to provide the optimum desired response (e.g., a therapeutic or prophylactic response). For example, a single dose or bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc., be proportionally reduced or increased as indicated by the exigencies of the therapeutic situation. In some instances, it may be especially advantageous to formulate compositions in dosage unit form for ease of administration and uniformity of dosage. The specification for the dosage unit forms are dictated by and directly dependent on (a) the unique characteristics of the active compound and the particular therapeutic or prophylactic effect to be achieved, and (b) the limitations inherent in the art of compounding such an active compound for graft protection.

Pharmaceutically acceptable salts, such as acid and base addition salts, are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids (e.g., hydrochloric or hydrobromic acid), sulfuric, nitric, phosphoric, and the like acids; or organic acids such as, for example, acetic, propanoic, hydroxyacetic, lactic, pyruvic, oxalic (e.g., ethanedioic), malonic, succinic (e.g., butanedioic acid), maleic, fumaric, malic (e.g., hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely the salt forms can be converted by treatment with an appropriate base into the free base form.

Compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, (e.g. the lithium, sodium, potassium, magnesium, calcium salts and the like), salts with organic bases, (e.g. the benzathine, N-methyl-D-glucamine, hydrabamine salts), and salts with amino acids such as, for example, arginine, lysine and the like. The term “addition salt” as used hereinabove also comprises the solvates which the compounds described herein are able to form. Such solvates are for example hydrates, alcoholates and the like.

The term “quaternary amine” as used hereinbefore defines quaternary ammonium salts which the compounds are able to form by reaction between a basic nitrogen of a compound and an appropriate quaternizing agent, such as, for example, an optionally substituted alkylhalide, arylhalide or arylalkylhalide, (e.g., methyliodide or benzyliodide).

Other reactants with good leaving groups may also be used, such as alkyl trifluoromethanesulfonates, alkyl methanesulfonates, and alkyl p-toluenesulfonates. A quaternary amine has a positively charged nitrogen.

Pharmaceutically acceptable counterions include chloro, bromo, iodo, trifluoroacetate, and acetate. The counterion of choice can be introduced using ion exchange resins.

As used herein, unless indicated otherwise, for instance in a structure, all compounds and/or structures described herein comprise all possible stereoisomers, individually or mixtures thereof. The compound and/or structure may be an enantiopure preparation consisting essentially of an (−) or (+) enantiomer of the compound, or may be a mixture of enantiomers in either equal (racemic) or unequal proportions.

A “group” or “functional group” is a portion of a larger molecule comprising or consisting of a grouping of atoms and/or bonds that confer a chemical or physical quality to a molecule. A “residue” is the portion of a compound or monomer that remains in a larger molecule, such as a polymer chain, after incorporation of that compound or monomer into the larger molecule. A “moiety” is a portion of a molecule, and can comprise one or more functional groups, and in the case of an “active moiety” can be a characteristic portion of a molecule or compound that imparts activity, such as pharmacological or physiological activity, to a molecule as contrasted to inactive portions of a molecule such as esters of active moieties, or salts of active agents.

As used herein, “alkyl” refers to straight, branched chain, or cyclic hydrocarbon groups including, for example, from 1 to 20 or more carbon atoms, for example and without limitation C₁₋₃, C₁₋₆, C₁₋₁₀ groups, for example and without limitation, straight, branched chain alkyl groups such as methyl, ethyl, propyl, butyl, pentyl, hexyl, heptyl, octyl, nonyl, decyl, undecyl, dodecyl, and the like. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. “lower alkyl” refers to C₁-C₆ alkyl. Non-limiting examples of straight alkyl groups include methyl, ethyl, propyl, butyl, pentyl, hexyl, heptyl, octyl, nonyl, and decyl. Branched alkyl groups comprises any straight alkyl group substituted with any number of alkyl groups. Non-limiting examples of branched alkyl groups include isopropyl, n-butyl, isobutyl, sec-butyl, and t-butyl. Non-limiting examples of cyclic alkyl groups include cyclopropyl, cyclobutyl, cyclopentyl, cyclohexyl, cycloheptlyl, and cyclooctyl groups. Cyclic alkyl groups also comprise fused-, bridged-, and spiro-bicycles and higher fused-, bridged-, and spiro-systems. A cyclic alkyl group can be substituted with any number of straight, branched, or cyclic alkyl groups. “Unsaturated alkyl” may comprise one or more, (e.g., 1, 2, 3, 4, or 5), carbon-to-carbon double bonds and alternatively may be referred to as alkene or alkenyl, as described below. “Substituted alkyl” can include alkyl substituted at 1 or more (e.g., 1, 2, 3, 4, 5, 6, or more) positions, which substituents are attached at any available atom to produce a stable compound, with substitution as described herein. “Optionally substituted alkyl” refers to alkyl or substituted alkyl. “Halogen,” “halide,” and “halo” refers to —F, —Cl, —Br, and/or —I. “Alkylene” and “substituted alkylene” can include divalent alkyl and divalent substituted alkyl, respectively, including, without limitation, methylene, ethylene, trimethylene, tetramethylene, pentamethylene, hexamethylene, hepamethylene, octamethylene, nonamethylene, or decamethylene. “Optionally substituted alkylene” can include alkylene or substituted alkylene.

The term “isostere”, also referred to as bioisosteres, refers to compounds, chemical substituents or groups with similar physical or chemical properties which produce broadly similar biological properties to another chemical compound. Isosteres have similar activity, e.g. heparanase-inhibitory activity in the context of the present disclosure. Isosteres often are pharmacophores; compounds comprising similar steric and electronic features. Suitable candidates for isosteres can be readily ascertained by medicinal chemists, and/or can be identified by in silico modeling.

Heparanase (HPSE) is an endo-beta-D glucuronidase that cleaves heparan sulfate in the endothelial glycocalyx. Inhibitors of heparanase (heparanase inhibitors) are a class of compounds effective to inhibit heparanase (see, e.g., Ding, J. (2011). Heparanase Inhibitors. In: Schwab, M. (eds) Encyclopedia of Cancer. Springer, Berlin, Heidelberg and Mohan C D, et al. Targeting Heparanase in Cancer: Inhibition by Synthetic, Chemically Modified, and Natural Compounds. iScience. 2019 May 31; 15:360-390). Heparanase inhibitors are known for their use in treatment of cancers (see, e.g., Tsunekawa N, et al. Heparanase augments inflammatory chemokine production from colorectal carcinoma cell lines. Biochem Biophys Res Commun. 2016 Jan. 22; 469(4):878-83).

Heparanase inhibitors are a well-known class of compounds of varying structure that are previously shown to be useful in treating cancers. Based on the studies below, heparanase inhibitors are useful in the graft/tissue/organ protection methods and compositions as described herein (See, Mohan C D, et al. iScience. 2019 May 31; 15:360-390, Ding, J. (2011). Heparanase Inhibitors. In: Schwab, M. (eds) Encyclopedia of Cancer. Springer, Berlin, Heidelbergfor listings and structures of exemplary heparanase inhibitors). Exemplary heparanase inhibitors include heparin, chemical derivatives of heparin, nonanticoagulant heparin, and other polyanionic molecules such as sulfated phosphomannopentaose (PI-88, Mupafostat), sulfated tri mannose C—C-linked dimers, trachyspic acid, trachyspic acid 19-butyl ester, oligomannurarate sulfate (JG3), SST0001 (Roneparstat), M402 (Necuparanib), laminaran sulfate, PG545 (Pixatimod) and its analogs (See, Mohan C D, et al. iScience. 2019 May 31; 15:360-390), and suramin (See, e.g., Ding, J. (2011). Heparanase Inhibitors. In: Schwab, M. (eds) Encyclopedia of Cancer. Springer, Berlin, Heidelberg). Other exemplary heparanase inhibitors include: 2-[4-propylamino-5-[5-(4-chloro)phenyl-benzoxazol-2-yl]phenyl]-2,3-dihydro-1,3-dioxo-1 H-isoindole-5-carboxylic acid, benzoxazol-5-ylacetic acids, RK-682 (3-hexadecanoyl-5-hydroxymethyltetronic acid), 1-[4-H-benzoimidazol-2-yl]-phenyl]-3-[4-(1 H-benzoimidazol-2-yl)-phenyl]-ureas such as 1,3-bis-[4-(1 H-benzoimidazol-2-yl)-phenyl]-urea, RK-682 series and the KI-105 series compounds, such as 4-Bn-RK-682, heparin and heparin sulfate-binding fragments of heparanase, Defibrotide, and RG-13577—polymerized from 4-hydroxyphenoxy monomers (MW ˜5,800). Antiheparanase antibodies, including antibody analogs such as scFv fragments, etc. also can inhibit heparanase activity. (See, e.g., Ding, J. (2011). Heparanase Inhibitors. In: Schwab, M. (eds) Encyclopedia of Cancer. Springer, Berlin, Heidelberg).

Heparanase inhibitors also include as a class, heparastatin and its analogs, including pharmaceutically-acceptable salts and isosteres (bioisosteres) thereof. Heparastatin (SF4, (3S,4S,5R,6R)-4,5-dihydroxy-6-[(2,2,2-trifluoroacetyl) amino]piperidine-3-carboxylic acid) is an example of a class of siastatin B-derived glycosidase inhibitors (see, e.g., U.S. Pat. No. 6,852,735; European Patent Publication No. EP 1197214 A1; Satoh T, et al. A practical synthesis from siastatin B of (3S,4S,5R,6R)-4,5-dihydroxy-6-(trifluoroacetamido)piperidine-3-carboxylic acid having antimetastatic activity in mice. Carbohydr Res. 1996 Jun. 5; 286:173-8; and Nishimura Y, et al. Flexible synthesis and biological activity of uronic acid-type gem-diamine 1-N-iminosugars: a new family of glycosidase inhibitors. Journal of Organic Chemistry 2000; 652-11). The following is an exemplary structure for heparastatin HCl:

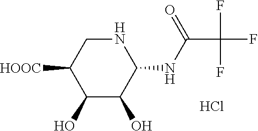 [SMILES: O=C(O)[C@H]1CN[C@H](NC(=O)C(F)(F)F)[C@@H](O)[C@H]1O ; Cl]

Heparanase inhibitors closely related to SF4/heparastatin include siastatin B derivatives having the exemplary structure:

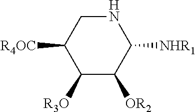

wherein:
R₁ is a hydrogen atom, an unsubstituted or halogen-substituted, straight or branched acyl group of 1-5 carbon atoms (C₁₋₅ acyl), e.g., a C₁₋₅ alkanoyl group, or —C(═NH)R₅ in which R₅ is a group —OR₆ or —NR₇R₈ where R₆, R₇, and Ra each stand for a hydrogen atom or a straight or branched alkyl group of 1-5 carbon atoms (C₁₋₅ alkyl);
R₂ and R₃ may be the same or different from each other and are a hydrogen atom or an unsubstituted or halogen-substituted, straight or branched C₁₋₅ acyl, e.g., a C₁₋₅ alkanoyl group; and
R₄ is —OR₉ or —NR₁₀R₁₁ where R₉, R₁₀, and R₁₁ each stand for, independently, a hydrogen atom or a straight or branched C₁₋₅ alkyl group,
   or a pharmacologically acceptable salt or isostere thereof. An acyl group is a group or moiety, such as R₁ above, generally having the structure:

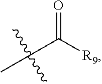

in which R₉ may be H, or a C₁₋₄ alkyl group in a C₁₋₅ acyl or C₁₋₅ alkanoyl group.

The heparanase inhibitor compound, e.g., heparastatin or a member of the above-described siastatin B derivatives, may be included in a pharmaceutical composition or drug product comprising the heparanase inhibitor compound and a pharmacologically acceptable carrier and/or excipient. See, e.g., EP 1197214 A1. The pharmaceutical composition or drug product may be in the form of a perfusate, for example comprising the heparanase inhibitor compound in an aqueous solution or emulsion. For example, the heparanase inhibitor may be dissolved in saline, water, phosphate-buffered saline (PBS), lactated Ringers, or any other suitable vehicle. The heparanase inhibitor is provided in a concentration effective to reduce transplant rejection of graft, (e.g., donor), tissue in a recipient patient in which the tissue is transplanted. The tissue may be an organ. The effective concentrations of the heparanase inhibitor in the perfusate solution can be determined empirically by in vitro, ex vivo, and in vivo testing according to standard practice in the medicinal arts. Exemplary concentrations of the heparanase inhibitor in the perfusate include: at least 10 nM, at least 50 nM, at least 100 nM, at least 500 nM, at least 1 μM, at least 10 μM, at least 25 μM, or at least 50 μM, including increments therebetween. The upper limits of the concentration of the heparanase inhibitor in the perfusate solution is limited only by toxic or deleterious effects of the composition, and can be determined empirically by in vitro, ex vivo, and in vivo testing according to standard practice in the medicinal arts, for example 100 μM, 1 mM, 10 mM. 100 mM, etc. Exemplary heparastatin concentrations useful for methods described herein range from 10 nM (nanomolar) to 1 mM (millimolar), or from 200 nM to 500 μM (micromolar), or at least 10 nM, at least 50 nM, at least 100 nM, at least 500 nM, at least 1 μM, at least 10 μM, at least 25 μM, or at least 50 μM and, optionally, no greater than 5 mM. Exemplary heparanase inhibitor concentrations useful for uses described herein include a therapeutic equivalent concentration to a concentration of SF-4 (heparastatin) ranging from 10 nM (nanomolar) to 1 mM (millimolar), or from 200 nM to 500 μM (micromolar), or at least 10 nM, at least 50 nM, at least 100 nM, at least 500 nM, at least 1 μM, at least 10 μM, at least 25 μM, or at least 50 μM and, optionally, no greater than 5 mM.

In use, the perfusate may be administered to the organ at any effective time(s) prior to, during, or after transplantation, for example: 1) by in situ perfusion of the tissue or organ while in the donor, 2) by ex vivo perfusion of the tissue or organ prior to implantation in the recipient, and/or 3) by administration or perfusion of the heparanase inhibitor to the recipient patient prior to, during, or after transplantation. As indicated, when administered to the recipient, the heparanase inhibitor may be administered to the patient systemically, as an intravenous product (e.g., as an infusion), or directly to the tissue or organ as a perfusate. As above, the heparanase inhibitor may be administered as a bolus, multiple times, continuously (e.g., as an infusion), intermittently, as needed based on any suitable clinical measure, or according to any other effective dosage regimen. The tissue may be perfused in the donor, then ex vivo, or may be perfused ex vivo, and the drug also can be administered to the recipient intravenously, or by perfusion. An organ may be catheterized in the recipient, allowing for local delivery to the organ, (e.g., perfusion), according to any suitable dosage regimen.

Heparanase inhibitors may be effectively delivered via perfusion, intravenous, inhalation, or oral administration to protect grafts on donors, ex vivo, and post-transplant recipients. While the Examples below describe the example of lung transplantation, the described approach can be broadly applicable for other solid organ transplantation and contribute to improving donor shortage, graft preservation, graft rejection, and post-transplant outcomes. As above, the described heparanase inhibitors may be formulated as an intravenous or perfusate product. The described heparanase inhibitors may be alternatively formulated as an inhaled or oral drug product for administration via oral, inhalation or nasopharyngeal delivery to a graft recipient according to any effective dosage and dosage regimen. A practitioner of ordinary skill can formulate suitable inhaled spray, aerosol, or powder formulations according to standard practice in the pharmaceutical arts for delivery to the respiratory tract. Likewise, oral drug products may be formulated according to any suitable delivery method, including immediate release, or delayed or enteric release. Any suitable delivery route and formulation may be used so long as the desired protective effect is achieved.

### EXAMPLES

The endothelial glycocalyx (eGC) is considered a key regulator of several mechanisms that prevent vascular injury and disease. Degradation of this macromolecular layer may be associated with post-transplant graft dysfunction. We demonstrated the benefits of eGC protection via heparanase inhibition on graft quality.

Using a rat model, in vivo warm ischemia induced heparanase activation leading the graft endothelial glycocalyx damage in the lungs. The ultrastructural changes of the eGC in lungs after 1-hour in vivo warm ischemia were confirmed by transmission electron microscopy. When we administer heparin to the rats intravenously before in vivo warm ischemia, the heparanase activity was decreased and the endothelial glycocalyx was preserved after the warm ischemic insult. To deny the effect from the heparin's anticoagulant property, we tested non-anticoagulant heparin (NAH) and obtained the similar observation, suggesting that the heparin and NAH's effects of heparanase inhibition solely preserved eGC during warm ischemia. To validate their transplantability, orthotopic lung transplant was performed. After transplant, lungs with damaged eGC exhibited impaired graft function, inflammation, edema, and inflammatory cell migration (e.g. polymorphonuclear neutrophils, T-cells and monocytes). However, lungs with preserved eGC via heparanase inhibition exhibited improved graft function and quality after transplantation.

Lung grafts were also subjected to normothermic ex vivo lung perfusion (EVLP) for detailed assessment under isolated conditions. Increased eGC shedding, that were determined by tissue-remaining glycosaminoglycans (GAGs) and perfusate syndecan-1 levels, was evident in the lungs with damaged eGC after reperfusion on EVLP. Endothelial dysfunction determined using Evans blue dye on EVLP was significantly increased in lungs with damaged eGC. Accordingly, pulmonary vascular resistance was markedly increased in the lungs with damaged eGC during EVLP. These reperfusion-related deficiencies were significantly attenuated in lungs with preserved eGC following heparanase inhibition. In addition to heparanase activity, the overall eGC degradation process undoubtedly involves the coordinated activation of multiple specific eGC shedding enzymes. The use of EVLP further revealed that activated MMP-2, a key enzyme involved in the proteolytic cleavage of eGC, increased in a time-dependent manner, resulting in prominent GAG and syndecan-1 degradation in lungs with damaged eGC following reperfusion. Our findings that pre-treatment with heparanase inhibitors effectively blocked GAG and syndecan-1 shedding and MMP-2 activation in lungs during subsequent reperfusion imply that activated heparanase or fragmented heparan sulfate could be a primary driving force for further eGC disintegration.

In summary, eGC vulnerability induced by ischemic damage directly resulted in inflammation, edema, and immune/inflammatory cell migration to the lungs after reperfusion in this rat model. Preserving the eGC by inhibiting heparanase improved graft function and reduced inflammation, suggesting the role of eGC in attenuating reperfusion injury in transplanted grafts. Consequently, structural preservation of the eGC may be a therapeutic strategy to improve post-transplant outcomes. Our findings suggest that heparanase inhibition could provide a novel therapeutic means for pulmonary endothelial protection leading to better graft preservation and post-transplant outcomes.

### Example 1

### Materials and Methods

Study design. To investigate the stability of eGC in lung grafts and its potential influence on graft function and quality after reperfusion, we established rat models with damaged or preserved lung eGC and performed orthotopic, single, left-lung transplantation or EVLP using the lungs from these rats. Animals were randomly assigned 4 different groups; (1) native lungs (normal control), (2) Control: lungs with eGC damage, (3) Heparin: lungs with eGC preserved by heparin, and (4) NAH: lungs with eGC preserved by NAH. Shedding of the eGC was induced in vivo by donor systemic ischemia. After 1 h of ischemia in vivo, lungs were procured in a standardized fashion for lung transplantation then either directly transplanted or subjected to EVLP and assessed biologically. Native lungs were not subjected to any insult.

Animals. Inbred male Lewis (RT-11) rats weighing 250-300 g were purchased (Harlan Sprague-Dawley Inc., Indianapolis, IN). Animals were maintained in laminar flow cages in a specific-pathogen-free animal facility at the University of Pittsburgh and fed a standard diet and water ad libitum.

Models of lungs with damaged or preserved eGC and rat orthotopic left lung transplantation. Rats were sedated with 4% isoflurane via inhalation for tracheostomy and then placed on a ventilator. They received 5% isoflurane with 100% O2 via inhalation through the ventilator for 15 min to induce a deep state of anesthesia that causes arrest of spontaneous breathing. The rats were disconnected from the ventilator 5 min after intravenous administration of heparin (300 IU), a heparin derivative without anti-coagulant properties (300 μg; N-acetyl heparin (NAH) (Millipore Sigma, Burlington, MA)), or saline (300 μl, control) through the jugular vein. Cardiac activity and blood oxygen saturation were monitored after disconnection from ventilator until cardiac arrest. One hour after induction of ischemia, a median thoracotomy was performed, and blood was flushed from the lungs with cold, low potassium dextran solution (PERFADEX; XVIVO Perfusion AB) through the pulmonary artery. Then, the heart-lung bloc was isolated and stored in cold PERFADEX for 6 h before transplantation.

Orthotopic single-lung transplantation of the left lung was performed using the cuff method. Two hours after reperfusion, the naive lung was clamped, 100% 02 was administered for 5 min through a ventilator, and the recipient's blood was sampled from the graft pulmonary vein for blood gas analysis.

Ex vivo lung perfusion in rats. EVLP was performed using a commercially available rodent EVLP system (IPL-2 Isolated Perfused Rat or Guinea Pig Lung System; Harvard Apparatus, Holliston, MA). After procurement, the lungs were kept under cold ischemia for 1 h, then placed on the EVLP system, ventilated with air warmed to 37′, and perfused with 100 ml STEEN solution (XVIVO Perfusion AB) that was deoxygenated with 6% O₂, 8% CO₂ and balanced N2 and supplemented with 50 mg of methylprednisolone (Solu-Medrol; Pfizer, Inc.) and 50 mg of cephalosporin (Cefazolin; West-Ward Pharmaceuticals Corp., Eatontown, NJ). Perfusion flow was started at 10% of target flow and gradually increased for 1 h toward a target flow rate that was calculated as 20% of cardiac output (75 ml/min/250 g donor body weight). Pulmonary artery pressure, peak airway pressure, and airway flow were monitored continuously, and lung compliance and PVR were calculated every hour.

Assessment of endothelial barrier function. After 3 h of EVLP, 6 mg of Evans blue dye (EBD, Millipore Sigma) was administered into the perfusate to assess vascular endothelial permeability. After 30 min of perfusion, the lung vascular bed was rinsed with 20 ml of PBS and pieces of lungs were incubated overnight in formamide (Millipore Sigma) at 60° C. Absorbance of the extractant was measured at 620 nm to quantitate the amount of EBD that had permeabilized into the tissue and was normalized to dry-tissue weight.

Wet-to-dry (W/D) weight ratio. The weight of the lung tissues was measured immediately after collection (wet weight). The lung tissue was then placed into a 60° C. oven to dry for 72 h. Tissues were weighed to deter-mine the dry weight, and the wet-to-dry ratio was calculated.

Real-time RT PCR. Lung tissues were collected after EVLP or 2 h after transplantation. Total RNA was extracted from the graft using TRIzol reagent (Life Technologies, Inc. Grand Island, NY) according to the manufacturer's instructions and purified by ethanol precipitation. RNA content was measured using 260/280 nm UV spectrophotometry. Messenger RNA (mRNA) levels for interleukin (IL)-6, IL-1p, metalloprotease (MMP)-9, and glyceraldehyde-3-phosphate dehydrogenase (GAPDH) were measured with SYBR Green 2-step, real-time reverse transcription-polymerase chain reaction (RT-PCR).

Western blotting. Whole cell lysate was isolated with 2% SDS buffer (2% SDS, 50 mM Tris-HCl pH 6.8, 10% glycerol) supplemented with protease inhibitors (Sigma-Aldrich). Membrane protein fractions were obtained with the Mem-PER Plus Membrane Protein Extraction Kit (Thermo Scientific) following manufacturer's instructions. Proteins were resolved on 10% SDS-PAGE followed by transfer to 0.2 μm nitrocellulose membranes (Bio-Rad Laboratories, Hercules, CA, USA). Membranes were blocked with 5% non-fat milk at room temperature for 2 h and then incubated with the following primary antibodies at 4° C. overnight: anti-syndecan-1 (1:1000; Santa Cruz Biotechnology, Inc., Dallas, Tx, USA), anti-heparanase-1 (1:1000; Boster Biological, Pleasanton, CA, USA), anti-VE-Cadherin (1:1000; Abcam), anti-HSP90 (1:1000; Abcam) and anti-@-actin (1:50,000; Sigma-Aldrich). Membranes were then incubated with either anti-mouse (1:5000) or anti-rabbit (1:3000) horse-radish peroxidase (HRP)-conjugated polyclonal secondary antibody (Abcam, Cambridge, MA, USA), or IRDye 800CW anti-rabbit IgG or 680CW anti-mice secondary antibody (LI-COR Biosciences, Lincoln, NE, USA) at room temperature for 2 h. Proteins were visualized using an enhanced chemiluminescence kit (Abcam), and protein bands were imaged and analyzed using Image Lab software (Version 6.0; Bio-Rad Laboratories, Inc.) or Odyssey 9120 Infrared Imaging System (LI-COR Biosciences).

Heparanase activity assay. Heparanase activity was measured using a heparanase assay kit (AMS Bio-technology (Europe) Ltd., Abingdon, UK) to quantitate heparan sulfate degradation according to the manufacturer's instructions. Briefly, isolated lung tissue lysate was applied to biotinylated heparan sulfate-coated wells and incubated with gentle shaking for 1 h at 37° C. After incubation and washing, the remaining heparan sulfate was detected using a biotin-streptavidin-HRP system.

Determination of glycocalyx components in rat lung grafts. For rat lung tissue, syndecan-1 released into the perfusate during EVLP was measured with a rat-specific ELISA (Immunotag Rat SDC1 (Syndecan-1) ELISA, G-Biosciences, St. Louis, MO). Glycocalyx glycosaminoglycans (GAGs) were quantitated using the 1,9-dimethylmethylene blue (DMMB, Millipore Sigma) method with modifications. A 1 L solution of DMMB (pH˜3.0) was prepared by dissolving 16 mg DMMB in distilled H2O containing 3.04 g/A glycine, 1.6 g/A NaCl and 95 ml 0.1 M acetic acid. Briefly, 1 ml DMMB solution was added to 20 μl lung tissue lysate then incubated at room temperature for 1 h on a tube rotator. The tubes were then centrifuged at 14,000 rpm for 15 min at 4° C. Supernatants were carefully decanted, 200 μL sodium lauryl sulfate buffer (2.08 mM) was added to each pellet, and the tubes were vortexed until GAG-DMMB pellets were no longer visible. GAG content in each sample was measured spectrophotometrically at 656 nm.

Gelatin zymography. Gelatin zymography was performed to measure secreted MMP-2 and MMP-9 activity in EVLP perfusate and to measure endogenous MMP-2 and 9 activity in lung tissue before transplantation. Briefly, lung tissue before transplant was homogenized in cold PBS. Perfusate samples (20 μl) or tissue lysates were mixed with 2× non-reducing SDS sample buffer were loaded onto a 10% polyacrylamide gel containing 0.1% SDS and 0.1% gelatin. After electrophoresis to separate the proteins, the gel was washed in renaturing buffer (Novex Invitrogen) at room temperature, and then incubated in developing buffer (Novex Invitrogen) at 37° C. overnight (˜16-18 h). The next day, the gel was stained with Coomassie Brilliant Blue R-250 (Bio-Rad) and imaged (ChemiDoc, Bio-Rad). Enzyme activity, visualized as clear bands against the dark blue background, was quantified using Image-J (NIH).

TEM for glycocalyx imaging. To prepare the tissue for transmission electron microscopy imaging, blood was flushed from rat lungs with PBS via the pulmonary artery after 1 h of warm ischemia, then the lungs were perfused with a solution containing 2% glutaraldehyde, 2% sucrose, 0.1 M sodium cacodylate buffer (pH 7.3, Sigma), and 2% lanthanum nitrate (Sigma). Excised lung tissues were incubated with the same solution for 2 h at 4° C. for fixation. The fixed tissues were immersed in a 2% sucrose, 0.1 M sodium cacodylate buffer (pH 7.3) solution with 2% lanthanum nitrate overnight at 4° C. to stain the glycocalyx, then washed twice with an alkaline solution (0.03 mol/l NaOH) containing 2% sucrose. Following fixation and glycocalyx staining with lanthanum nitrate, the specimens were rinsed in PBS, post-fixed in 1% osmium tetroxide (Electron Microscopy Sciences, Hatfield, PA), rinsed in PBS a second time, dehydrated through a graded series of ethanol and propylene oxide (Electron Microscopy Sciences), and embedded using a Poly/Bed 812 (Luft formulations) Embedding Kit (Polysciences, Warrington, PA). Sections were cut on a Leica-Reichart Ultracut ultra-microtome (Leica Microsystems, Buffalo Grove, IL). Semi-thin (300 nm) sections were stained with 0.5% toluidine blue in 1% sodium borate (toluidine blue O and sodium borate, Fisher Scientific, Pittsburgh, PA) and examined under the light microscope. Ultrathin sections (65 nm) were stained with uranyl acetate and Reynold's lead citrate and examined on a JEOL 1400 transmission electron microscope (JEOL Peabody, MA) with a side-mount AMT 2 k digital camera (Advanced Microscopy Techniques, Danvers, MA).

Tissue staining and histopathological analysis. Formalin-fixed, paraffin-embedded lung tissues collected 2 h after transplantation were sectioned to 4-μm thickness and stained with hematoxylin and eosin. The sections were also stained for immunofluorescent imaging using primary antibodies for CD3 (Invitrogen), CD68 (Invitrogen), or syndecan-1 (Santa Cruz), and Hoechst 33342 dye for nuclear staining. Primary antibody was detected by secondarily Cy5-conjugated goat anti-rabbit IgG (Invitrogen) and Cy3-conjugated goat anti-mouse IgG (Millipore Sigma).

Polymorphonuclear neutrophils (PMNs) were stained using a naphthol AS-D chloroacetate esterase staining kit (Millipore Sigma) and identified by nuclear morphology staining in bright red. Stained slides were scanned with a whole-slide image scanner (Axio Scan.Z1; Carl Zeiss) and analyzed with digital image processing software (ZEN lite blue edition; Carl Zeiss). Stained cells were counted by two investigators with the identity of the samples masked.

Statistical analysis. All data were analyzed using SPSS Version 25 statistical software package (SPSS Inc., Chicago, IL). The data are considered as continuous variables with approximately normal distributions and presented as mean±standard deviation with individual data plots. The data were analyzed with one-way analysis of variance (ANOVA) followed by post hoc analysis with the Bonferroni correction for multiple comparisons. Data from multiple observations over time were analyzed using 2-way repeated measures ANOVA. A probability level of p<0.05 was considered statistically significant.

### Results

Confirmation of an established Intragraft endothellal glycocalyx damage model. To perform this study, we first established an animal model of eGC damage in lung grafts induced by ischemic insult in vivo.

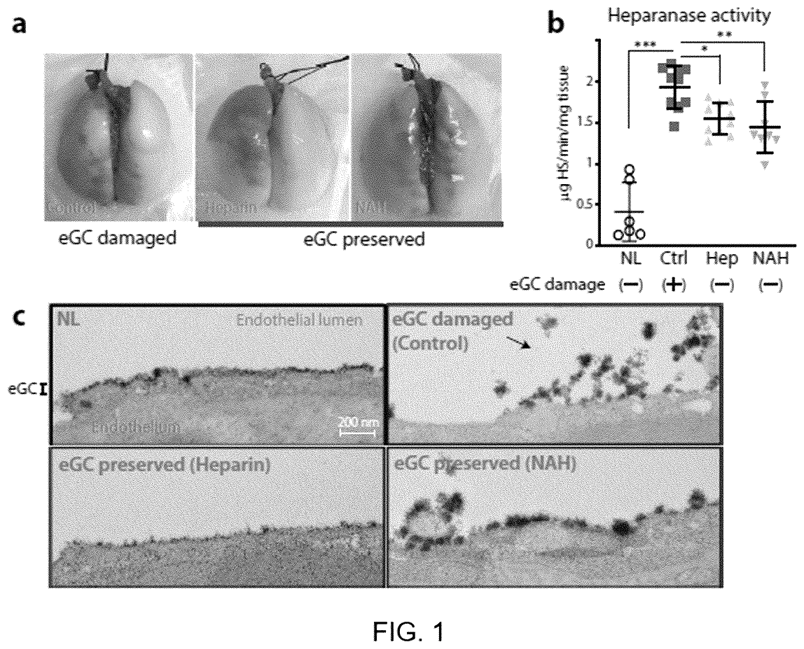

After cardiopulmonary arrest was induced in donor rats with an anesthesia overdose, the donor was kept at room temperature for an hour to induce ischemic insult in the lungs. In some rats, heparin, a clinically available heparanase inhibitor, was administered intravenously via the jugular vein 5 min before the ischemic insult with the purpose of preserving the eGC from enzymatic degradation by heparanase. Other rats received N-acetyl heparin (NAH), a heparin-derivative that lacks anticoagulant activity, which allowed us to confirm that heparin's effects on the eGC were independent of its anti-coagulation activity. After 1 h of in vivo ischemic insult at room temperature, lungs were procured in a standardized fashion for lung transplantation. Gross visual inspection of excised lungs revealed no significant changes in appearance following heparin or NAH treatment (FIG. 1 (a)). However, heparanase activity was significantly elevated in lungs with no treatment and was inhibited by both heparin and NAH (FIG. 1 (b)) No differences in heparanase protein expression in the lung tissues were noted among the groups.

Transmission electron microscopy (TEM) revealed ultra-structural changes in the eGC in the lung vasculature after warm ischemic insult. Fixation buffer containing 2% lanthanum, which specifically binds to the glycocalyx, was perfused via the pulmonary artery during graft procurement to label and visualize the eGC under TEM. In control lungs, the eGC was desquamated from the endothelial cell surface with complete dissociation in certain regions. In contrast, the eGC was intact and adherent to the cell surface in lungs pre-treated with either heparin or NAH prior to ischemic insult, similar to the eGC morphology observed in native lungs (FIG. 1 (c)). After establishing these experimental models of lungs with damaged (control; no treatment) or preserved eGC (heparin/NAH treated) regulated by heparanase activity, we began to investigate how the status of intragraft eGC affects post-transplant graft quality in lung transplantation.

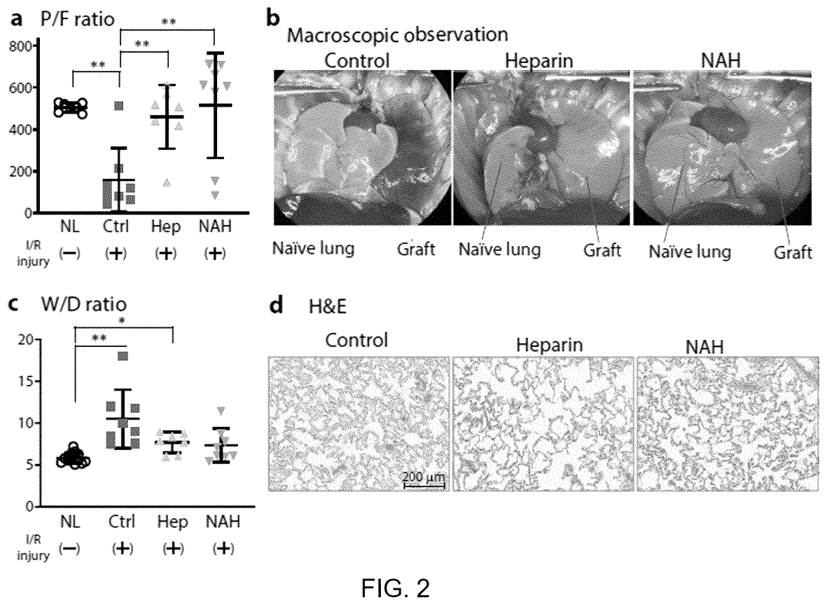

Physiological performance and immune cell infiltration after transplant in lung grafts with damaged endothelial glycocalyx. To examine the impact of eGC damage on graft quality after trans-plantation using our established models, we performed rat orthotopic single lung transplantation using a 3-cuff technique. Lung grafts in all groups were subjected to cold ischemia at 4° C. for 6 h, then transplanted into syngeneic recipients and re-perfused for 2 h to simulate a clinical scenario. Two hours after reperfusion, the gas exchange ability of the lung grafts was evaluated by gas analysis of blood in the pulmonary vein of the graft during single-lung ventilation with 100%02. Lungs in control exhibited impaired gas exchange function as indicated by a reduced PaO2/FiO2 (P/F) ratio 2 h after reperfusion, when compared with native lungs. Conversely, lung grafts with preserved eGC due to heparin or NAH treatment displayed markedly improved post-transplant function, as indicated by a significantly higher P/F ratio 2 h after transplantation as compared with control lung grafts (FIG. 2 (a)). Reperfusion-induced pulmonary edema was clearly evident in lungs without heparin/NAH pre-treatment, both macroscopically (FIG. 2 (b)) and microscopically (FIG. 2 (d)) and was objectively quantified by measuring the wet-to dry (W/D) ratio. Control lungs after transplant had significant congestion, swollen alveolar walls, and secretions in the airways, and the W/D ratio was twice that observed in native lung samples (p<0.01) (FIG. 2 (c)). In contrast, grafts with preserved eGC after either heparin or NAH administration exhibited less pulmonary edema, which was reflected in a lower W/D ratio 2 h after transplantation. Significant congestion, swollen alveolar walls, and secretions in the airways were not observed in lungs with preserved eGC after heparanase inhibition by heparin/NAH (FIG. 2 (d)).

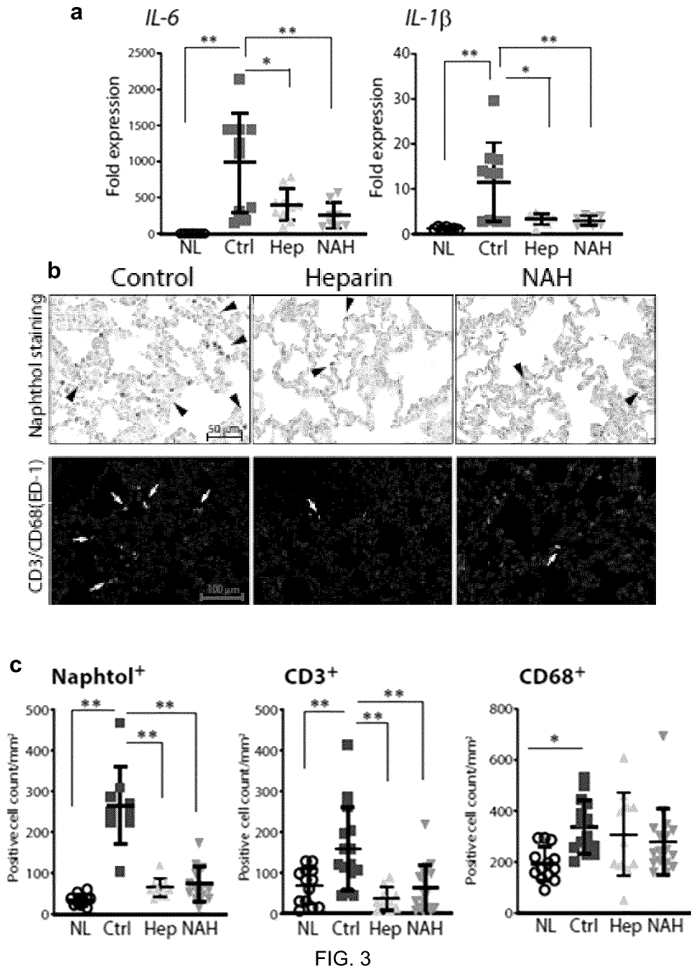

The expression profiles of mRNAs for proinflammatory cytokines within the graft tissues 2 h after reperfusion were examined using quantitative real-time reverse-transcription polymerase chain reaction (RT-PCR). Proinflammatory cytokine expression of both interleukin (IL)-6 and IL-1β were higher in control lung grafts than in native lungs (FIG. 3 (a)). In contrast, mRNA expression levels of IL-6 and IL-1β were significantly lower in grafts treated with heparin/NAH as compared with lung grafts in control. In addition, we found that reduced cellular infiltration was directly associated with eGC preservation after heparanase inhibition. Specifically, graft-tissue staining indicated significantly reduced polymorphonuclear neutrophils (PMNs) (naphthol-positive staining), T-cell (CD3+-positive immunostaining) and macrophage (CD68+-positive immunostaining) infiltration in lungs with preserved eGC by heparin/NAH administration as compared with control lung grafts 2 h after reperfusion (FIG. 3 (b,c)).

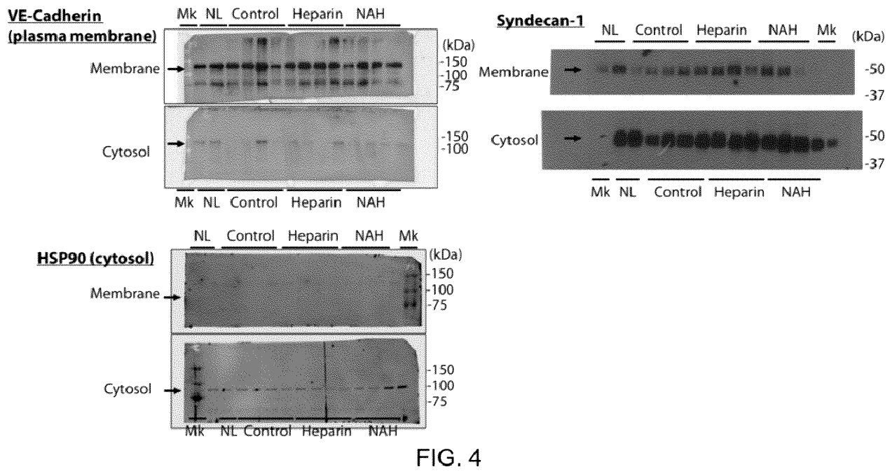

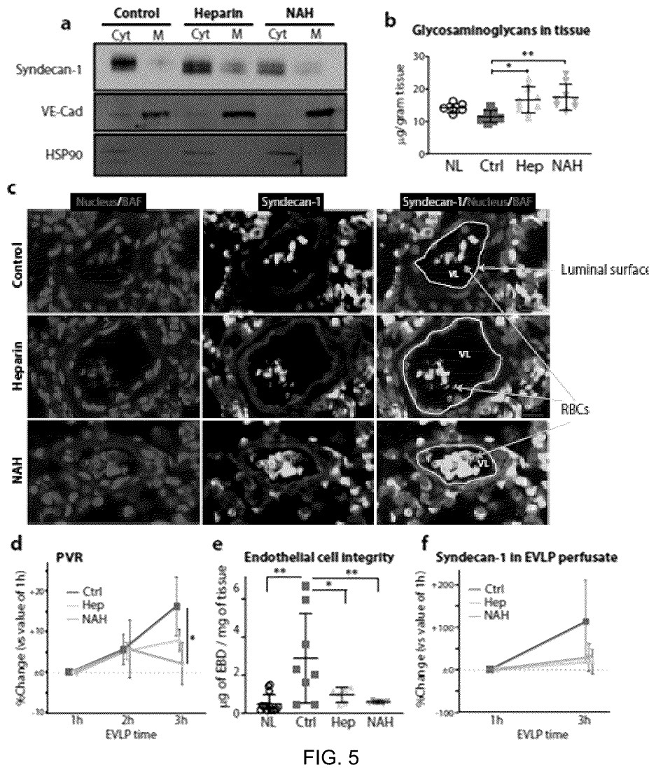

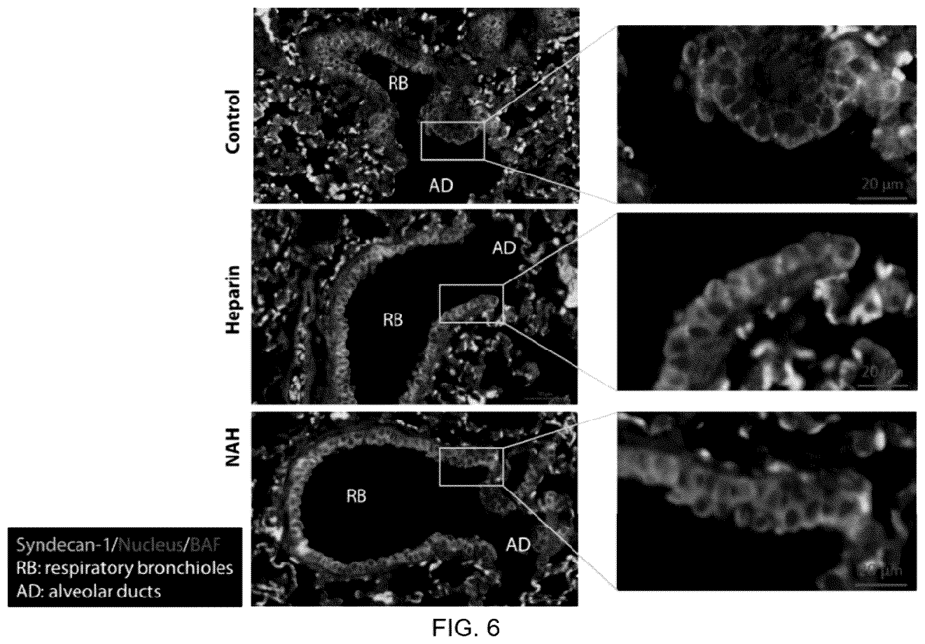

Further investigation of eGC shedding and endothelial damage after transplantation in the lungs with intact versus damaged endothelial glycocalyx. After demonstrating changes in tissue physiology and extravasation of inflammatory cells in grafts during reperfusion dependent on the integrity of the eGC, we next analyzed eGC integrity in lung grafts after transplantation. Membrane and cytosolic proteins fractions were isolated from lung tissues after transplantation to quantitate the remaining glycoproteins on the plasma membrane by western blotting and compare glycoprotein differences between grafts with and without pre-transplant heparanase inhibition. Successful separation and isolation of membrane and cytosolic fractions from tissue samples was confirmed by western blotting for vascular endothelial (VE)-cadherin and heat shock protein 90 (HSP90) as loading markers for plasma membrane protein fractions and cytosol protein fractions, respectively (FIG. 4). Syndecan-1 (a major eGC proteoglycan) protein expression in the plasma membrane was markedly higher 2 h after transplantation in heparin/NAH treated lung grafts as compared with control lung grafts (FIG. 5 (a)). Similarly, 1,9-dimethylmethylene blue (DMMB)-based GAG quantification assays revealed that significantly more GAGs remained in the lung tissue 2 h after trans-plant in grafts with heparin/NAH preserved eGC as compared with control lungs grafts with no eGC protection (FIG. 5 (b)). Immunofluorescent (IF) staining for syndecan-1 in lung grafts 2 h after reperfusion was performed to assess localization of syndecan-1 in peripheral vasculature and airway and to clarify whether syndecan-1 was lost from the vascular endothelium. Staining was assessed in the peripheral lung vasculature (φ˜50 μm) and in the pulmonary epithelium in the respiratory bronchioles near the alveolar ducts (φ˜100 μm). If staining clearly showed loss of syndecan-1 expression in the endothelial lumen of the peripheral vasculature in the transplanted control lung tissue with no protection of eGC as compared with transplanted lungs with preserved eGC (heparin or NAH-treated) (FIG. 5 (c)). In contrast, syndecan-1 was intact on the endoluminal portion of the pulmonary epithelium where respiratory bronchioles transitioned to alveolar ducts in the transplanted lung tissue, and no difference was observed among treatment groups (FIG. 6).

To investigate the dynamic changes in the vasculature and endothelial barrier function of lungs during reperfusion in an isolated environment, we performed normothermic acellular EVLP on the rat lungs. To specifically focus on the effects of eGC status, we minimized cold ischemic time to 1 h before placing the lung grafts to EVLP. Lungs from the damaged-eGC and preserved-eGC rat models were perfused with acellular STEEN solution and ventilated on the system at normothermia for 3 h. The control lungs exhibited a significant increase in pulmonary vascular resistance (PVR) with time during EVLP, while lungs with preserved eGC with donor heparin/NAH treatment had little to no increase in PVR while on EVLP (FIG. 5 (d)). Endothelial barrier integrity was quantified by examining Evans blue dye (EBD) penetration into the lung parenchyma during the last 30 min of EVLP. The lungs in control showed significantly increased penetration of EBD as compared with native lungs. In contrast, both heparin-treated and NAH-treated lungs displayed significantly less EBD penetration (FIG. 5 (e)). Furthermore, we found that syndecan-1 concentrations in the EVLP perfusate were maintained at a constant level during EVLP of lungs with heparin/NAH preserved eGC as compared with increasing levels of syndecan-1 in the perfusate from lungs in control (FIG. 5 (f)).

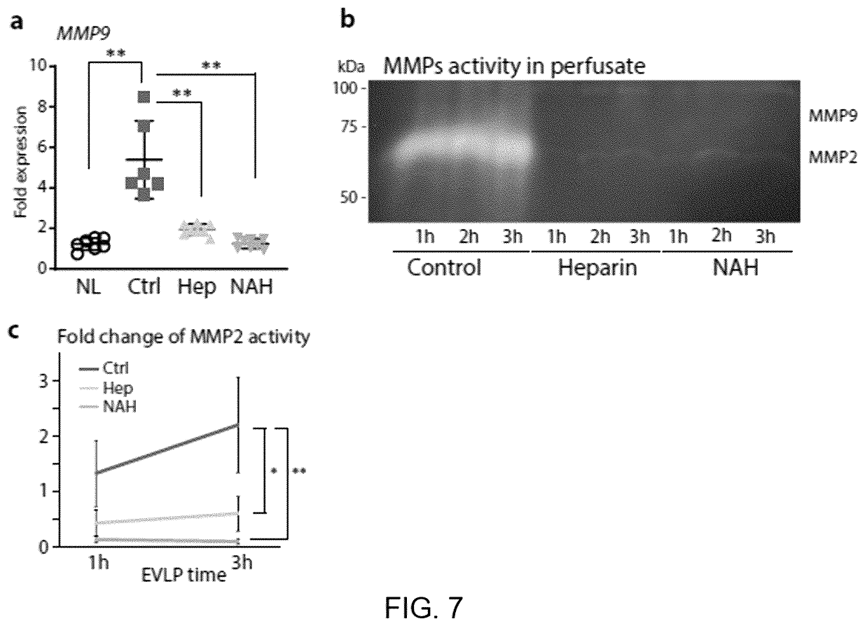

In addition to heparanase, other proteolytic enzymes play an important role in regulating homeostatic eGC shedding, and MMP-2 and MMP-9 are known to cleave syndecan-1 at the cell membrane. Accordingly, we examined the mRNA expression profiles of MMP-9 in lung tissue after transplant using quantitative RT-PCR. MMP-9 mRNA expression was significantly lower in grafts treated with heparin/NAH as compared with lung grafts in control 2 h after transplantation (FIG. 7 (a)). To further confirm these findings, we measured secreted MMP-2 and MMP-9 enzymatic activity in the EVLP lung perfusate using gelatin zymography. In control lungs, MMP-2 activity in the perfusate increased significantly over time. In stark contrast, MMP-2 activity in EVLP perfusate from lungs with heparin/NAH pretreated was significantly less than that seen in controls with eGC damage and remained unchanged during EVLP (FIG. 7 (b,c)). The activity of MMP-9, which predominantly exists in inflammatory cells (e.g. neutrophils) was low and did not changed during EVLP, and no significant differences in MMP-9 activity were observed among groups (FIG. 7 (b)). This suggests that MMP-2 may uniquely facilitate eGC shedding after reperfusion. Gelatin zymography using tissue lysates supported this finding and showed that endogenous MMP2 was predominantly expressed in pre-transplant lung tissue regardless of the status of the eGC, suggesting that the observed glycocalyx shedding during EVLP primarily resulted from tissue-specific factors. Overall, these findings suggest that donor lungs with preserved eGC display significantly reduced tissue-specific MMP-2 protein activity.

### Example 2

The effects of inhibiting heparanase (HPSE) on lung quality during EVLP is evaluated. Human clinical EVLP perfusate from lung graft patients was utilized to identify a potential association between glycocalyx integrity in grafted lung tissue and clinical data. In addition, pre-clinical studies were performed in which rat lungs underwent normothermic EVLP for 4 hours with/without HPSE inhibitors, heparin (1000-U/hour) or heparastatin (SF4; 1-μM), added to the perfusate. After 4-hours EVLP, left lungs were transplanted into syngeneic rats then evaluated for graft quality 2-hours after reperfusion. Clinically, increased degradation of syndecan-1 was identified in dysfunctional grafts during EVLP. Levels of heparan sulfate in perfusate after EVLP were associated with incidence of graft dysfunction after transplantation. In the pre-clinical rat study, SF4 effectively inhibited HPSE activity, and significantly attenuated dissociated glycocalyx levels, endothelial dysfunction, edema, and inflammation in lungs during EVLP compared to both controls and heparin groups. High-doses of heparin demonstrated markedly increased perfusate syndecan-1 concentrations and deteriorated lung quality during EVLP compared with controls. Post-transplant graft function and inflammation were significantly improved in SF4-treated group compared to those in both control and heparin-treated groups. This study demonstrated that HPSE activity inhibition by SF4 can improve graft preservation during EVLP by protecting the glycocalyx and endothelial function, leading to better lung function following transplantation.

### Materials and Methods

Clinical EVLP and lung transplantation: Clinical EVLP was performed with XPS machine and STEEN solution (XVIVO Perfusion AB, Denver, CO) for lung grafts with marginal quality from death donors. Perfusate samples from clinical EVLP cases were collected and kept in −80° C. Glycocalyx perfusate components, heparan sulfate and syndecan-1, were detected by ELISA as described below. PGD grade were determined through routine clinical evaluation according to criteria of the International Society for Heart and Lung Transplantation.

Detection of glycocalyx shedding in lung allografts during clinical EVLP: To determine the degree of glycocalyx shedding in lung grafts, we measured syndecan-1 and heparan sulfate in the perfusate at 1 hour and at the end of EVLP. The concentrations of syndecan-1 and heparan sulfate in clinical EVLP perfusate were determined by enzyme-linked immunosorbent assay (ELISA, human syndecan-1 ELISA kit: Abcam, Cambridge, UK; heparan sulfate ELISA kit: Biotang Inc., Waltham, MA) as per the manufacturer's instructions. Syndecan-1 and heparan sulfate concentrations were normalized to donor ideal body weight (iBW), multiplied by the volume of perfusate to calculate the absolute amount, and divided by EVLP run time to calculate release rates. The data were compared between the grafts declined and accepted for transplantation after EVLP. Also, the potential association between glycocalyx shedding and severe PGD incidence at 72 hours posttransplant were analyzed among transplanted lungs.

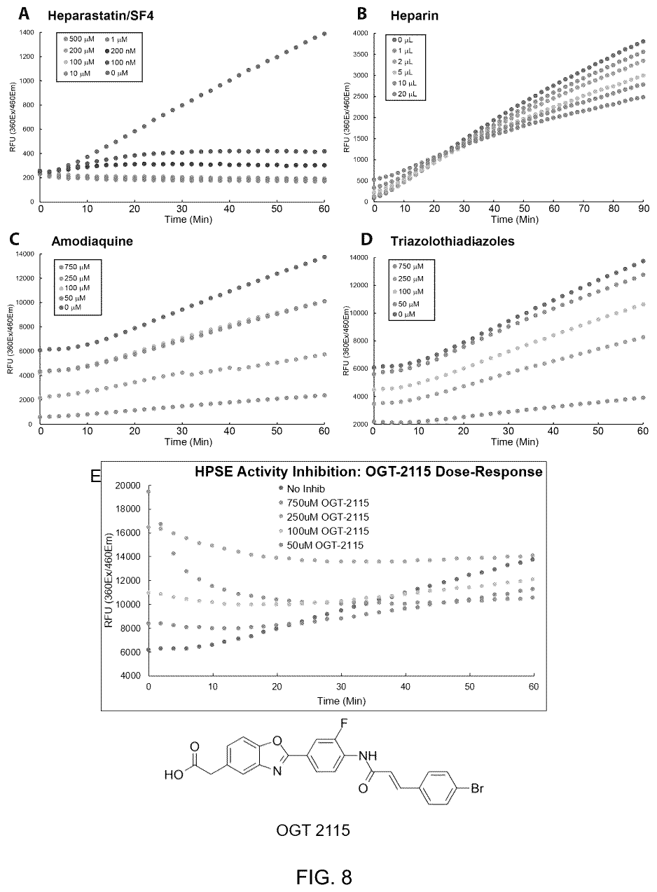

Animal study design: Potential HPSE inhibitors including SF4 were initially tested in-vitro using human lung microvascular endothelial cells (HLMVECs) to define dose, toxicity, and suitability for organ preservation (see below). To investigate the impact of HPSE inhibition on graft quality during EVLP using lungs from Inbred male Lewis (RT-1^{I}) rats (250-300 g, Envigo RMS, Inc., Indianapolis, IN.), heparin and heparastatin (SF4) hydrochloride ((3S,4S,5R,6R)-4,5-dihydroxy-6-(2,2,2-trifluoroacetamido) piperidine-3-carboxylic acid; DiagnoCine LLC, Hackensack, NJ), an iminosugar-based HPSE inhibitor, were administered into the EVLP perfusate. In the heparin group, 1000-U of heparin were initially added to the EVLP perfusate, then 1000-U of heparin were administered every hour during EVLP. This protocol was established based upon our previous dosing studies and on the biological half-life of. SF4 was administered into the perfusate to a final concentration of 1 μM prior to EVLP. This concentration of SF4 was found to be sufficient to effectively inhibit HPSE during 4-hours EVLP based on our preliminary dose-dependent cytotoxicity studies with cultured HLMVECs (FIG. 8). After 4-hours EVLP, right lungs were used for molecular biology analyses of mRNA/protein/enzyme activity (Table A), and left lungs were used for lung transplant. Syngeneic rats were used as recipients and graft quality was evaluated 2-hours after transplantation. Two control conditions were included in the experimental protocol: 1) sham controls (Sham) were lungs not placed on EVLP and 2) untreated controls (Control) were lungs placed on EVLP without HPSE inhibitor treatment. EVLP physiology, endothelial integrity, inflammation and glycocalyx preservation for EVLP lungs and post-transplant lung grafts were investigated across all treatment regimens.

TABLE A
Relative mRNA expressions (compared to control) of cell injury markers in the human
lung microvascular endothelial cells after treating heparanase inhibitor candidates.

| | | | | | | | | Cell viability |
|---|---|---|---|---|---|---|---|---|
| | HO-1 | IL6 | TNF-α | MCP1 | Bcl-2 | Bax | Bcl-xl | (~%) |
| Control | 1.01 | 1.00 | UD | 1.06 | UD | 1.06 | 1.05 | 100% |
| SF4 | 1.02 | 0.53 | UD | 1.02 | UD | 0.85 | 0.50 | 100% |
| Triazolothiadiazoles | 3.15 | UD | UD | 9.89 | UD | 1.28 | UD | 10% |
| Amodiaquine | 2.94 | 13.55 | UD | 3.45 | UD | 0.71 | 1.30 | 10% |

Bax: B-cell lymphoma 2 Associated X,
Bcl-2: B-cell lymphoma 2,
Bcl-xL: B-cell lymphoma-extra-large,
HO-1, hemeoxygenatse-1;
IL-6, interleukin 6;
SF4: Heparastatin,
TNF-α: tumor necrosis factor-α;
UD: undetermined

In-vitro cellular toxicity test for HPSE Inhibitor candidates: Human lung microvascular endothelial cells (HLMVECs) were obtained from the American Type Culture Collection (Rockville, MD, USA). Cells were cultured in a humidified incubator at 37° C. and 5% CO₂ and grown in endothelial growth medium (EGM™-2 Endothelial Cell Growth Medium-2, Lonza, Basel, Switzerland) supplemented with 2% heat-inactivated fetal bovine serum and 0.04% hydrocortisone. Cell media was replaced every 48 hours, and the cells were split upon 80-90% confluence.

For determination of HPSE inhibitor cellular cytotoxicity, HLMVECs were first incubated with HPSE inhibitors (100 nM SF4, 250 mM amodiaquine, 250 mM triazolothiadiazoles) and 5% DMSO (control). Final tested concentrations of HPSE inhibitors were calculated based upon repeatable in vitro dose-response studies. After 24 hours incubation, cell viability was confirmed by Trypan blue exclusion assay. Total mRNA was isolated using Trizol (Thermo Fisher Scientific Inc., Waltham, MA) and relative messenger RNA (mRNA) expression levels of hemeoxygenase-1 (HO-1), interleukin (IL)-6, tumor necrosis factor (TNF)-α, monocyte chemoattractant protein (MCP)-1, B-cell lymphoma 2 Associated X (Bax), B-cell lymphoma 2 (Bcl-2), B-cell lymphoma-extra-large (Bcl-xL) were quantified by SYBR Green 2-step, real-time, reverse transcription-polymerase chain reaction (RT-PCR). Relative gene expression levels were normalized to mRNA expression of glyceraldehyde-3-phosphate dehydrogenase (GAPDH) and compared to relative mRNA expression levels in control-treated HLMVEC.

In vitro β-D-glucuronidase activity assay Heparanase activity was measured via β-Glucuronidase activity assay using 4-methylumbelliferyl-β-D-glucuronide (4-MUG) substrate (Millipore-Sigma). As a source of enzymes, platelet rich plasma (PRP) obtained from rat whole blood or rat lung whole tissue lysate, was used. In brief, PRP or lysate was incubated with 10 μM 4-MUG at 37° C., including various doses of several heparanase (HPSE) inhibitors including triazolothiadiazoles (Chevron Science Center, Department of Chemistry, University of Pittsburgh, PA), amodiaquine (Sigma), heparin and heparastatin (SF4; Diagnocin LLC., Hackensack, NJ), and OGT-2115. These compounds have been previously described in cancer research as effective HPSE inhibitors. Fluorescence (λex 360 nm/λem 460 nm) was continuously measured for 60 minutes using a fluorescence microplate reader (BioTech Synergy HTX, Agilent Technologies, Santa Clara, CA).

Statilsticai analysis: All data were analyzed using SPSS Version 25 statistical software (SPSS Inc., Chicago, IL). Results were expressed as mean±standard deviation (SD). Data comparing two groups was analyzed by Mann Whitney U test, whole multiple observations over time were analyzed using two-way repeated-measures analysis of variance (ANOVA) and post hoc analysis was performed with the Turkey HSD test for multiple groups. Other data involving multiple groups were analyzed with one-way ANOVA followed by post hoc analysis with the Bonferroni correction for multiple comparisons. A probability level of p<0.05 was considered statistically significant.

Ex vivo lung perfusion in rats: EVLP was performed using a commercially-available rodent EVLP system (IL-2 Isolated Perfused Rat or Guinea Pig Lung System; Harvard apparatus, Holliston, MA). Acellular Steen solution was used for EVLP perfusate and medicated with methylprednisolone (Solu-Medrol®; Pfizer, Inc., New York, NY) and cephalosporin (Cefazolin; WG critical care LLC, Paramus, NJ) equally in all experimental groups. Perfusion flow was started at 10% of target flow and gradually increased for 1 hour toward a target flow rate that was calculated as 20% of cardiac output (75 ml/min/250 g donor body weight). Pulmonary artery pressure, peak airway pressure, and airway flow were monitored continuously, and dynamic lung compliance (Cdyn) and pulmonary vascular resistance (PVR) were analyzed. When assessing the PaO₂/F_{I}O₂ ratio (P/F ratio), the ex vivo perfused lungs were ventilated with 100% 02 for 5 minutes, and then the perfusate was sampled for blood gas analysis.

Rat orthotopic left lung transplantation following EVLP: Orthotopic, single-lung transplantation of the left lung was performed using the 3-cuff method. After EVLP for 4 hours, the lungs were cooled with 4° C. Perfadex on the EVLP system and stored at 4° C. for 1 hour prior to transplantation. The recipient animals were sacrificed 2 hours after reperfusion. When analyzing graft function, the native lung was clamped, 100% 02 was administered for 5 minutes through a ventilator, and the recipient's blood was sampled from the graft pulmonary vein for blood gas analysis.

Evaluation of endothellal barrier Integrity After 4 hours of EVLP, 60 mg of Evans blue dye (EBD, Millipore-Sigma, Burlington, MA) was administered into the perfusate to assess vascular endothelial permeability. After 30 minutes of perfusion, the lung vascular bed was rinsed with 20 ml of phosphate-buffered saline (PBS), and slices of lungs were incubated in formamide (Millipore Sigma) overnight at 60° C. Absorbance of the extractant was measured at 620 nm, and EBD was normalized to dry-tissue weight.

Wet-to-Dry (W/D) weight ratio: Lung tissues collected 2 hours after transplant were immediately weighed then placed in a 60° C. oven to dry for 72 hours. Tissues were weighed again after drying to determine the wet-to-dry lung weight ratio.

Real-time RT PCR: The levels of messenger RNA (mRNA) were assessed using SYBR Green 2-step, real-time, reverse transcription-polymerase chain reaction (RT-PCR). Graft tissues collected after 4 hours of EVLP and 2 hours after reperfusion were examined for relative mRNA expression levels of interleukin (IL)-6, IL-1, tumor necrosis factor (TNF)-α, and glyceraldehyde-3-phosphate dehydrogenase (GAPDH). Gene expression levels were normalized to GAPDH mRNA levels.

Western blotting: Total isolated proteins were extracted/resolved by 10% SDS-PAGE followed by transfer to 0.2 μm nitrocellulose membranes (Bio-Rad Laboratories, Hercules, CA, USA). Membranes were blocked with 5% non-fat milk at room temperature for 2 hours and then incubated overnight at 4° C. with the following primary antibodies: inhibitor of KB-α (IκB-α; #9242, Cell Signaling Technology, Inc., Danvers, MA) and β-actin (#A1978, Millipore-Sigma). Membranes were then incubated with either goat anti-mouse IgG (H+L) (#31430, Thermo Fisher Scientific Inc.; 1:5000) or goat anti-rabbit IgG (H+L) (#31460, Thermo Fisher Scientific Inc.; 1:3000) horseradish peroxidase-conjugated polyclonal secondary antibodies at room temperature for 2 hours. Proteins were visualized using an enhanced chemiluminescence kit (Abcam), and protein bands were imaged and analyzed using Image Lab software (Version 6.0; Bio-Rad Laboratories, Inc., Hercules, CA). The intensities of protein bands were quantitated by Image J software (National Institutes of Health, Bethesda, MD).

Syndecan-1 ELISA: Syndecan-1 concentrations in EVLP perfusate were quantitated using ELISA (Immunotag™ Rat Syndecan-1 ELISA, G-Biosciences, Geno Technology, Inc., St. Louis, MO). ELISA was performed according to the manufacturer's instructions using perfusate samples acquired after 4 hours of EVLP.

HPSE activity assay. HPSE activity was measured via a β-glucuronidase activity assay using 4-methylumbelliferyl-β-D-glucuronide (4-MUG) substrate (Millipore-Sigma). Briefly, after 4 hours of EVLP, lung tissues were homogenized in cold PBS. Then, 15 μL of isolated protein lysate was incubated with 10 μM 4-MUG at 37° C. in a total reaction volume of 100 μL. Fluorescence (λex 360 nm/λem 460 nm) was continuously measured for 60 minutes using a fluorescence microplate reader (BioTech Synergy HTX, Agilent Technologies, Santa Clara, CA).

Determination of glycosaminoglycans (GAGS): The concentration of GAGs in rat EVLP perfusate at 4 hours was quantitated using 1,9-dimethylmethylene blue (DMMB, Millipore Sigma). The DMMB solution (pH˜3.0) was prepared by dissolving 16 mg/I DMMB in distilled H₂O containing 3.04 g/I glycine, 1.6 g/l NaCl and 95 ml 0.1 M acetic acid. Briefly, 1 ml DMMB solution was mixed with 100 μl EVLP perfusate then incubated for 18 h at room temperature. The tubes were then centrifuged at 14,000 rpm for 20 min at 4° C. Supernatants were carefully decanted, 100 μl dissolvent buffer (4 M guanidine hydrochloride, 50 mM sodium acetate, 10% propan-1-ol, pH 6.8) was added to each pellet, and the tubes were incubated at 40° C. and vortexed until GAG-DMMB pellets were completely dissolved. GAG content in each sample was measured spectrophotometrically at 656 nm.

Gelatin zymography: Gelatin zymography was performed to measure secreted MMP-2 and MMP-9 activities in EVLP perfusate. Briefly, 20 μl of perfusate was mixed with 2×SDS sample buffer then loaded onto a 10% polyacrylamide gel containing 0.1% SDS and 10% gelatin. After protein separation by electrophoresis, the gel was washed in renaturing buffer (Novex™ Invitrogen™) at room temperature, and then incubated in developing buffer (Novex™ Invitrogen™) overnight at 37° C. The next day, the gel was stained with Coomassie Brilliant Blue R-250 (Bio-Rad) and imaged (ChemiDoc, Bio-Rad). Enzyme activity, visualized as clear bands against the dark blue background, was quantified using Image-J (NIH).

Tissue staining and histopathological analysis: Formalin-fixed, paraffin-embedded lung tissues collected 2 hours after transplantation were sectioned to 4 μm thickness and stained with hematoxylin and eosin. In parallel, sections of tissue collected after 4 hours of EVLP were stained for immunofluorescence imaging using primary antibodies for syndecan-1 (DL-101) (#sc-12765, Santa Cruz) and caveolin-1 (#3238, Cell Signaling Technology, Danvers, MA) and with Hoechst 33342 dye (Thermo Scientific). Donkey Cy3-conjugated anti-mouse IgG (#AP192C, MilliporeSigma, Burlington, MA) and Cy5-conjugated goat anti-rabbit IgG (#A10523, Thermo Scientific) secondary antibodies were used to detect primary antibodies. Stained slides were scanned with a whole-slide image scanner (Axio Scan.Z1; Carl Zeiss AB, Oberkochen, Germany) and analyzed with digital image processing software (ZEN lite blue edition; Carl Zeiss).

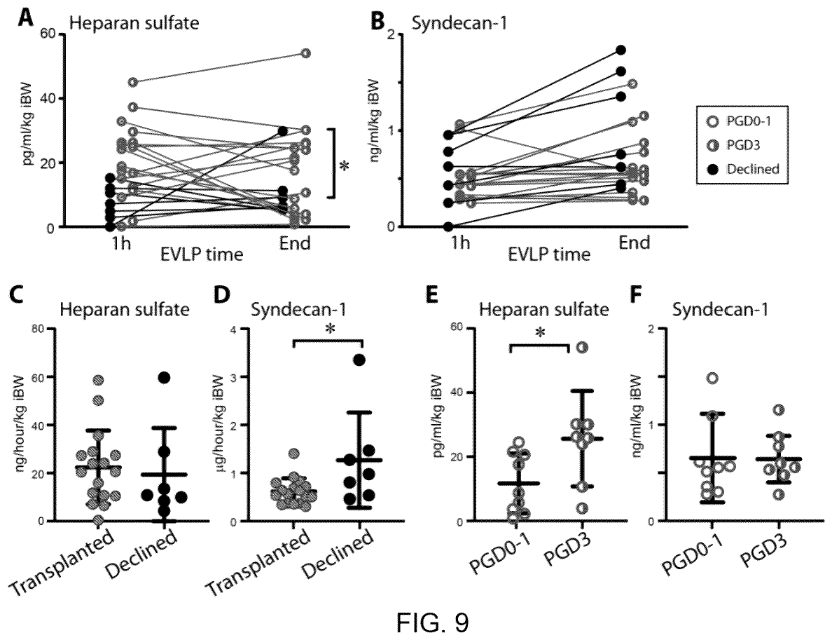

The association between human lung glycocalyx shedding during EVLP and graft transplantability and PGD: Human perfusate samples were utilized to assess the extent of glycocalyx shedding in 24 lung allografts during clinical EVLP. Most declined lungs developed significant edema bilaterally or lobar-localized. FIG. 9 (A,B) shows the time dependent changes of heparan sulfate and syndecan-1 concentration in perfusate for lungs declined for transplant, developed PGD0-1 and PGD after transplant. Regarding glycocalyx stability, the degradation of heparan sulfate over time during EVLP was similar between transplanted and declined lungs (FIG. 9 (C)); however, significantly higher perfusate levels of syndecan-1 accumulated in declined lungs compared to lungs transplanted after EVLP. (FIG. 9 (D)) In addition, as a secondary assessment tool to define the predictive value of glycocalyx degradation on post-transplant outcomes, we investigated the potential correlation between glycocalyx content at the end of EVLP and severe PGD incidence at 72 hours after EVLP/lung transplantation. Among transplanted lungs, lungs that developed PGD grade 3 (PGD3) after EVLP/transplant presented with higher perfusate levels of heparan sulfate at the end of EVLP compared to PGD0-1 lungs. (FIG. 9 (A,E)) Syndecan-1 levels in the perfusate at the end of EVLP were not associated with PGD3 (FIG. 9 (B,F)).

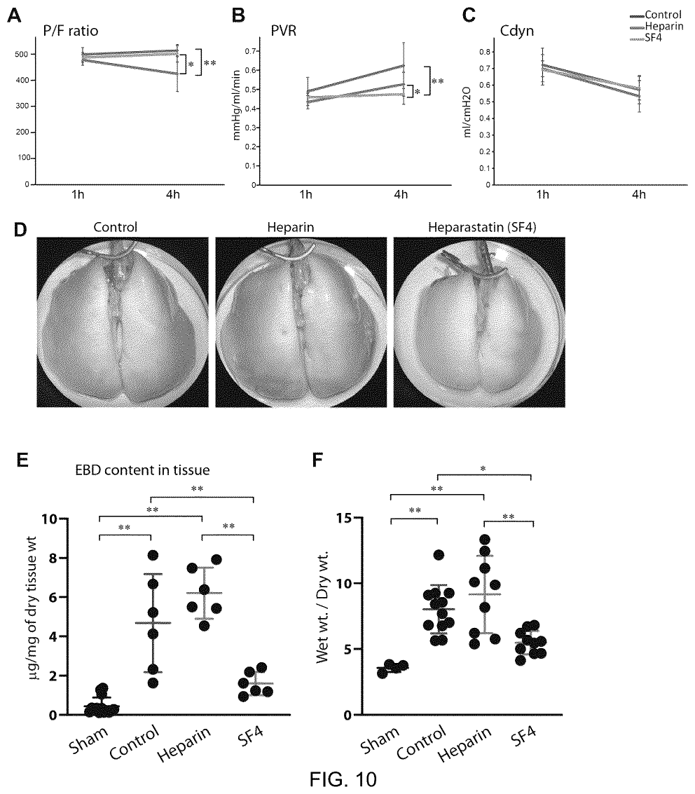

Administration of heparastatin (SF4) Improves rat lung endothellal preservation during EVLP Considering the increased glycocalyx breakdown in declined and PGD3 associated human lungs during EVLP, we sought to examine the potential effects of SF4 on preserving lung vascular endothelium and graft quality during EVLP in rats. Based on our preliminary in vitro cytotoxicity data and on published half-lives, we administered high-doses of heparin and a single-dose of SF4 in rat perfusate to sufficiently inhibit lung HPSE activity over the course of 4-hour EVLP. Control lungs (no HPSE inhibitor) displayed stable physiologic parameters including pulmonary vascular resistance (PVR) and dynamic lung compliance (Cdyn) during EVLP. In addition, rat lungs from both HPSE inhibitor groups, (heparin and SF4) possessed stable PVR and Cdyn during EVLP. However, we observed increased PVR in heparin-treated group. SF4 administration stabilized lung PVR compared to both control and heparin groups. Furthermore, neither heparin nor SF4 altered Cdyn during EVLP compared to control-treated lungs (FIG. 10 (A-C)).

Endothelial barrier function was grossly assessed by visualizing Evans blue dye (EBD) accumulation in rat lungs after EVLP. Compared to SF4-treated lungs, lungs in controls and heparin-treated groups displayed visibly increased EBD staining, suggesting impaired endothelial barrier function. (FIG. 10 (D)) When EBD content was quantified in the lungs after 4-hours EVLP, significantly more EBD had accumulated in untreated (control) and heparin-treated lungs as compared with sham lungs, and EBD accumulation was markedly decreased by SF4 treatment, supporting the visual findings. (FIG. 10 (E)) Measuring the W/D ratio revealed increased edema in control lungs, consistent with decreased endothelial barrier function, which was further augmented by heparin administration. SF4 significantly decreased the W/D ratio as compared with the control and heparin-treated lungs, indicating less edema (FIG. 10 (F)).

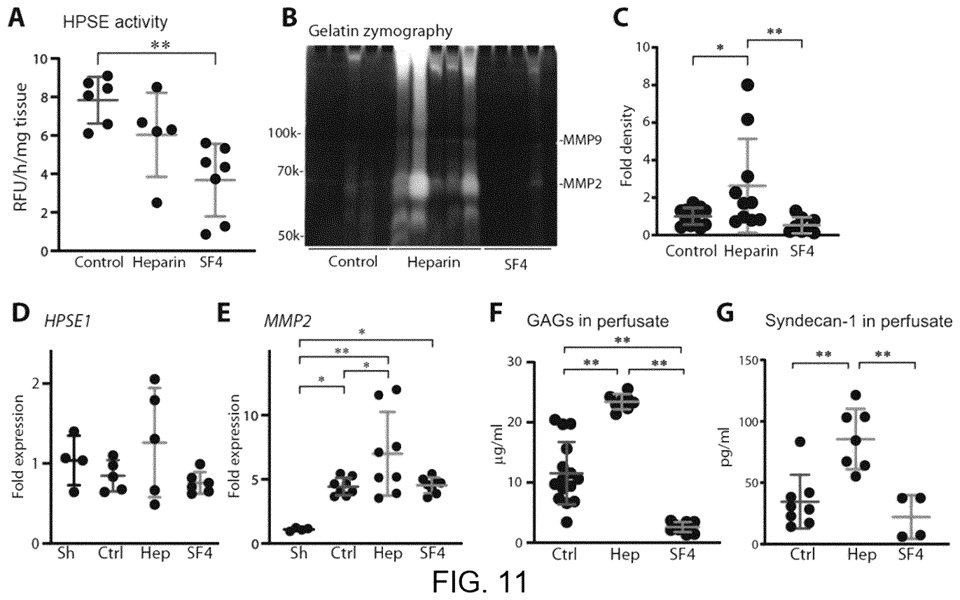

The effects of heparin and SF4 on glycocalyx-degrading enzymes: HPSE activity in the lungs after 4-hours EVLP was significantly inhibited by SF4 administration and partially inhibited by heparin administration. (FIG. 11 (A)) In addition, zymography analysis of EVLP perfusate revealed significantly elevated MMP-2 activity in the heparin group when compared to both the SF4 and control groups. (FIG. 11 (B,C) ) The mRNA expression of HPSE1 was not altered by either heparin or SF4 treatment as compared to sham. (FIG. 11 (D)) In contrast, gene expression of pro-MMP2 was upregulated during EVLP as compared with sham, and further upregulated by heparin administration. (FIG. 11 (E))

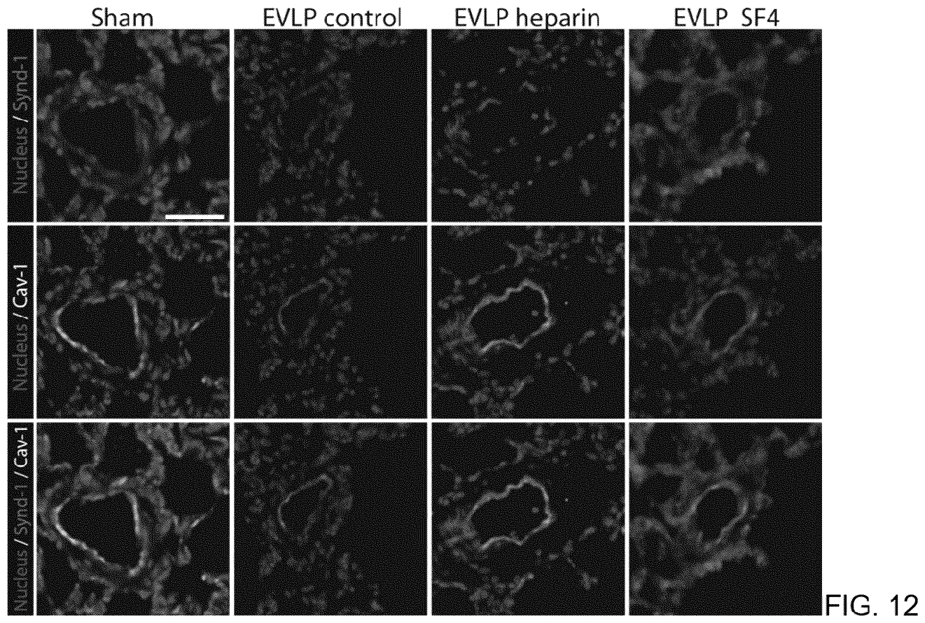

To determine the extent of glycocalyx shedding in rat lungs during EVLP, perfusate levels of GAGs and syndecan-1 were measured. SF4 administration significantly reduced the content of GAGs in EVLP perfusate at 4-hours EVLP compared to controls. (FIG. 11 (F)) In addition, SF4-treated lungs possessed significantly lower syndecan-1 perfusate levels versus heparin-treated lungs. (FIG. 11 (G)) As a note, the GAGs assay mechanism can detect heparin therefore the value in the heparin group is not comparable even though the statistically highest value among groups. Furthermore, concentrations of syndecan-1 in the EVLP perfusate after 4 hours was significantly higher in the heparin group as compared with controls. In Consistent with GAG perfusate data, immunofluorescent staining for syndecan-1 of lung endothelial lumen revealed significantly increased staining in the SF4-treated group compared to both the controls and heparin-treated groups after 4-hours EVLP (FIG. 12).

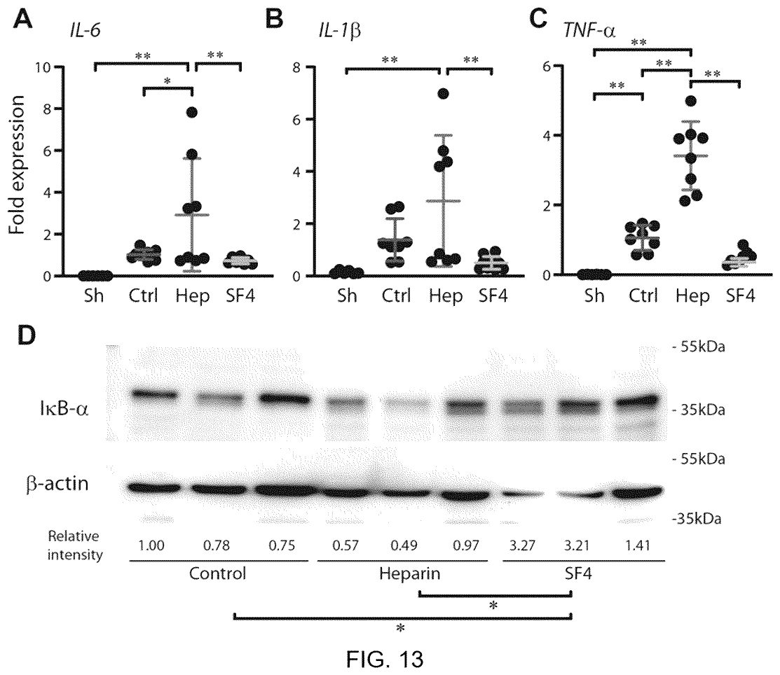

HPSE Inhibition downregulated NF-κB signaling in rat lungs during EVLP: Next, we examined the effects of HPSE inhibition on pro-inflammatory signaling in the lungs among the treatment groups after 4-hours EVLP. In control lung tissues, 4-hours EVLP increased mRNA expression for IL-6, IL-1p, and TNF-α as compared to sham lungs. Surprisingly, heparin treatment elicited increased mRNA expression of these cytokine genes versus both sham and control lungs. In contrast, SF4 treatment significantly downregulated the mRNA expression of these proinflammatory cytokines in rat lungs after EVLP as compared to lung mRNA expression levels in both heparin-treated and control groups. (FG. 13 (A-C)) Interestingly, SF4 significantly stabilized IκB-α expression in the lung tissue after 4-hours EVLP compared to control and heparin groups (FIG. 13 (D)).

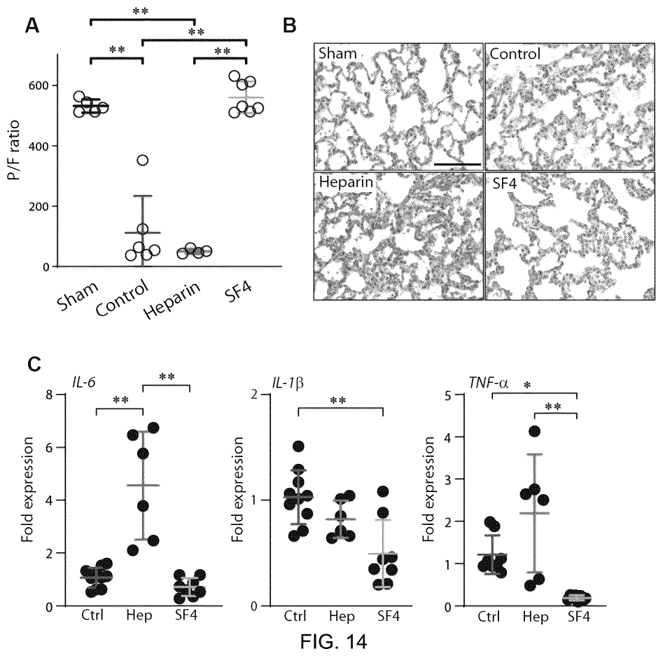

SF4-Induced HPSE Inhibition and endothellal preservation Improved graft function and quality after transplant: Finally, early post-transplant graft function was assessed in rat lungs placed on EVLP with and without HPSE inhibitors. Lung function, as measured by the P/F ratio, was significantly lower in recipients of untreated controls and heparin-treated grafts, when compared to recipients of sham and SF4 groups (FIG. 14 (A)). H&E staining of untreated controls and heparin-treated grafts revealed swollen alveolar walls, congestion in the blood vessels, and fluid leaks into the alveolar space. Conversely, SF4-treated lungs showed reduced swollen alveolar walls, congestion in the blood vessels, and fluid leaks (FIG. 14 (B)). The mRNA expression of proinflammatory cytokines (IL-6, IL-1β, and TNF-α were significantly lower in SF4-treated lungs 2 hours after transplant as compared with untreated control lungs and heparin-pretreated lungs (FIG. 14 (C)).

Adequate endothelial preservation is necessary for effective organ preservation. Sufficient evidence exists demonstrating a clear association between endothelial damage, graft quality and PGD after lung transplant. The therapeutic role of glycocalyx preservation to improve post-transplant outcomes has also been well-recognized. However, effective therapies for glycocalyx preservation during lung transplantation are still investigational. Here, it is observed that human lung glycocalyx degradation measured in perfusate during clinical EVLP is associated with the decision not to proceed with transplant due to deteriorating lung quality. Consistent with our findings in human lungs, our animal data also suggests that elevated perfusate syndecan-1 levels are associated with graft function and edema development during EVLP. In addition, our data shows that enhanced heparan sulfate levels in perfusate at the end of EVLP can potentially serve as a predictor of severe PGD after transplant.

Heparan sulfate suppresses the shedding of proteoglycans and structurally supports glycocalyx integrity. The loss of heparan sulfate from the core endothelial proteins results in increased shedding of proteoglycans such as syndecan-1. Therefore, therapies aimed at preserving endothelial heparan sulfate, and thus glycocalyx integrity, during EVLP may maintain graft quality and potentially improve post-transplant outcomes. The effects of HPSE inhibition on graft endothelial glycocalyx preservation during EVLP and post-transplant outcomes using a small animal model were investigated. Similar to clinical findings, standard EVLP conditions (no HPSE inhibition) caused increased lung endothelial permeability and GAGs shedding, resulting in poor post-transplant graft function. Notably, HPSE activity inhibition by SF4 resulted in improved structural integrity of endothelial glycocalyx and overall lung graft quality and function.

HPSE Inhibition as an endothelial glycocalyx preservation strategy for graft preservation during EVLP: Normothermic EVLP conditions are known to activate cellular metabolism, cellular synthesis and enzymatic activities in lung grafts. Here, it is found that HPSE and MMP2 enzymes are activated in lungs during EVLP and can trigger or exacerbate shedding of the endothelial glycocalyx. Several EVLP-associated factors can be considered as triggers of HPSE activation. Progressive acidosis in the perfusate is frequently observed during EVLP using acellular Steen solution, which was designed for short-term perfusion and has limited buffering capacity. Additionally, pro-inflammatory and hypoxia signaling can be induced, as perfusion may be heterogeneously distributed within the lungs during EVLP. Taken together, we believe these factors can activate HPSE and increase the burden on the vascular endothelium leading to increased susceptibility of lung grafts to cellular damage during perfusion and subsequent reperfusion after transplantation. In this animal study, we discovered that administration of SF4 can effectively reduce HPSE activity and help maintain the endothelium in rat lungs during EVLP. As a result of SF4 treatment, these lung grafts exhibited stable PVR and attenuated edema development. Interestingly, we found that lung compliance did not change by SF4 treatment, perhaps suggesting that an alternative airway protection strategy may be required to further improve organ preservation during EVLP.

Several HPSE inhibitors have been developed as potential therapeutics for cancer. While most of them exhibit cellular toxicity that precludes their use during EVLP, our preliminary investigations with endothelial cell cultures found that both heparin and SF4 are safe. SF4 is a derivative of gem-diamine1-N-iminosugars (new class of glycosidase inhibitors) able to inhibit HPSE in cancer studies (see, Nishimura Y, et al. Journal of Organic Chemistry 2000; 65:2-11; Sue M, et al. An iminosugar-based heparanase inhibitor heparastatin (SF4) suppresses infiltration of neutrophils and monocytes into inflamed dorsal air pouches. Int Immunopharmacol. 2016 June; 35:15-21; and Satoh T, et al. Carbohydrate Research 1996; 286:173-8). Our preliminary dose-response findings showed that SF4 has a very potent inhibitory effect against HPSE even at very low concentrations (<1 μM) with virtually no toxicity against HLMVECs, suggesting a potential for clinical translation.

The administration of heparin is another viable option for HPSE inhibition. Although heparin was added to the perfusate in clinical EVLP studies, glycocalyx degradation was still observed and associated with poor organ function, as we found in this study. In agreement with these findings, in vitro data provided herein suggests that heparin inhibition of HPSE is not as effective as SF4. Although it was found that the anti-HPSE efficacy of heparin increased in a dose-dependent manner, this necessitates a higher dose required to obtain a therapeutic benefit. Based on in vitro HLMVEC data, a 10× higher dose of heparin was used in rat EVLP (50 U/ml) compared to 5 U/ml routinely used for current clinical EVLP. Nonetheless, it was found that HPSE activity was not sufficiently nor significantly inhibited in lung tissue during EVLP. Also, an increase in both mRNA expression and extracellular release of active MMP2 was observed, culminating in glycocalyx damage rather than its preservation. Indeed, several previously published in vitro/in vivo studies have demonstrated that heparin can impair nitric oxide production in endothelial cells, perhaps mediated via glycocalyx injury. This may help explain the observed PVR increase in rat lungs during EVLP with heparin administration. Additionally, heparin facilitates the hydrolysis of tissue inhibitors of MMPs, thus allowing MMPs to remain active. Our study provides insights on the benefits of HPSE activity inhibition with SF4. HPSE inhibition with maintenance of glycocalyx structure can lead to better organ preservation and potentially rescue some organs during EVLP contributing to an increase in the donor pool while improving post-transplant outcomes.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A method of preparing graft tissue for transplantation, comprising contacting the tissue with an amount of a heparanase inhibitor effective to reduce transplant rejection of the tissue in a recipient patient in which the tissue is transplanted.

2. The method of claim 1, wherein the heparanase inhibitor comprises:
a. a compound selected from the group consisting of: heparin, chemical derivatives of heparin, nonanticoagulant heparin, sulfated phosphomannopentaose (PI-88, Mupafostat), sulfated tri mannose C—C-linked dimers, trachyspic acid, trachyspic acid 19-butyl ester, oligomannurarate sulfate (JG3), SST0001 (Roneparstat), M402 (Necuparanib), laminaran sulfate, PG545 (Pixatimod) and its analogs, 2-[4-propylamino-5-[5-(4-chloro)phenyl-benzoxazol-2-yl]phenyl]-2,3-dihydro-1,3-dioxo-1 H-isoindole-5-carboxylic acid, benzoxazol-5-ylacetic acids, RK-682 (3-hexadecanoyl-5-hydroxymethyltetronic acid), 1-[4-H-benzoimidazol-2-yl]-phenyl]-3-[4-(1 H-benzoimidazol-2-yl)-phenyl]-ureas such as 1,3-bis-[4-(1 H-benzoimidazol-2-yl)-phenyl]-uma, RK-682 series compounds such as 4-Bn-RK-682, KI-105 series compounds, heparin and heparin sulfate-binding fragments of heparanase, defibrotide, RG-13577, and an antiheparanase antibody, or a pharmacologically acceptable salt or isostere of any of the preceding:
b. the compound having the structure: 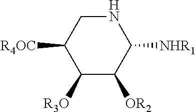
wherein:
R₁ is a hydrogen atom, an unsubsituted or halogen-substituted, straight or branched acyl group of 1-5 carbon atoms (C₁₋₅ acyl), or —C(═NH)R₅ in which R₅ is a group —OR₆ or —NR₇R₈ where R₆, R₇, and R₈ each stand for a hydrogen atom or a straight or branched alkyl group of 1-5 carbon atoms (C₁₋₅ alkyl);
R₂ and R₃ may be the same or different from each other and are a hydrogen atom or an unsubstituted or halogen-substituted, straight or branched C₁₋₅ acyl; and
R₄ is —OR₉ or —NR₁₀R₁₁ where R₉, R₁₀, and R₁₁ each stand for, independently, a hydrogen atom or a straight or branched C₃₋₅ alkyl group,
or a pharmacologically acceptable salt or isostere thereof,
wherein optionally R₁, R₂, and/or R₃ are, independently a C₁₋₅ alkanoyl group; or
c. a heparastatin, or a pharmaceutically-acceptable salt or isostere thereof, e.g., heparastatin HCl having the exemplary structure;

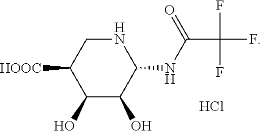 [SMILES: O=C(O)[C@H]1CN[C@H](NC(=O)C(F)(F)F)[C@@H](O)[C@H]1O ; Cl]

3-5. (canceled)

6. The method of claim 1, wherein the tissue is contacted with the heparanase inhibitor while the tissue is in a donor of the tissue or the tissue is contacted with the heparanase inhibitor ex vivo or in vitro.

7. (canceled)

8. The method of claim 1, wherein the tissue is contacted with the heparanase inhibitor after implantation in a recipient of the tissue.

9. The method of claim 1, wherein the tissue is in the form of an organ and wherein the organ optionally is perfused with a perfusate comprising the heparanase inhibitor.

10. (canceled)

11. The method claim 1, wherein the tissue is contacted with a solution of the heparanase inhibitor in which the concentration of the heparanase inhibitor, e.g., heparastatin, ranges from 10 nM (nanomolar) to 5 mM (millimolar).

12. (canceled)

13. The method of claim 11, wherein the tissue is contacted with the be heparanase inhibitor in a single bolus, or in multiple doses, intermittently, or continuously over a time period ranging from 10 minutes, 20 minutes, 30 minutes, 45 minutes, 1 hour, 2 hours, 3 hours, 4 hours, 5 hours, 6 hours, 12 hours, or 24 hours, or any increment therebetween.

14. The method of claim 1, wherein the tissue comprises lung tissue.

15. (canceled)

16. The method of claim 1, wherein the heparanase inhibitor is contacted with the tissue at a therapeutic equivalent concentration to a concentration of SF-4 (heparastatin) of at least 10 nM, at least 50 nM, at least 100 nM, at least 500 nM, at least 1 μM, at least 10 μM, at least 25 μM, or at least 50 μM and, optionally, no greater than 5 mM.

17. Graft tissue, such as lung tissue or a lung, prepared according to the method of claim 1.

18. A method for transplanting graft tissue from a donor patient to a recipient patient, comprising, implanting the tissue into the recipient patient, and prior to or after transplanting the tissue into the recipient, contacting the tissue with an amount of a heparanase inhibitor compound effective to reduce transplant rejection of the tissue in a recipient patient in which the tissue is transplanted.

19. The method of claim 18, wherein the heparanase inhibitor compound comprises:
a. a compound selected from the group consisting of: heparin, chemical derivatives of heparin, nonanticoagulant heparin, sulfated phosphomannopentaose (PI-88, Mupafostat), sulfated tri mannose C—C-linked dimers, trachyspic acid, trachyspic acid 19-butyl ester, oligomannurarate sulfate (JG3), SST0001 (Roneparstat), M402 (Necuparanib), laminaran sulfate, PG545 (Pixatimod) and its analogs, 2-[4-propylamino-5-[5-(4-chloro)phenyl-benzoxazol-2-yl]phenyl]-2,3-dihydro-1,3-dioxo-1 H-isoindole-5-carboxylic acid, benzoxazol-5-ylacetic acids, RK-682 (3-hexadecanoyl-5-hydroxymethyltetronic acid), 1-[4-H-benzoimidazol-2-yl]-phenyl]-3-[4-(1 H-benzoimidazol-2-yl)-phenyl]-ureas such as 1,3-bis-[4-(1 H-benzoimidazol-2-yl)-phenyl]-urea, RK-682 series compounds such as 4-Bn-RK-682, KI-105 series compounds, heparin and heparin sulfate-binding fragments of heparanase, defibrotide, RG-13577, and an antiheparanase antibody, or a pharmacologically acceptable salt or isostere of any of the preceding:
b. a compound having the structure: 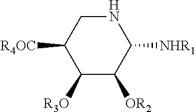
wherein:
R₁ is a hydrogen atom, an unsubsituted or halogen-substituted, straight or branched acyl group of 1-5 carbon atoms (C₁₋₅ acyl), or —C(═NH)R₅ in which R₅ is a group —OR₆ or —NR₇R₈ where R₆, R₇, and R₈ each stand for a hydrogen atom or a straight or branched alkyl group of 1-5 carbon atoms (C₁₋₅ alkyl);
R₂ and R₃ may be the same or different from each other and are a hydrogen atom or an unsubstituted or halogen-substituted, straight or branched C₁₋₅ acyl; and
R₄ is —OR₉ or —NR₁₀R₁₁ where R₉, R₁₀, and R₁₁ each stand for, independently, a hydrogen atom or a straight or branched C₃₋₅ alkyl group,
or a pharmacologically acceptable salt or isostere thereof,
wherein optionally R₁, R₂, and/or R₃ are, independently a C₁₋₅ alkanoyl group; or
c. a heparastatin, or a pharmaceutically-acceptable salt or isostere thereof, e.g., heparastatin HCl having the exemplary structure;

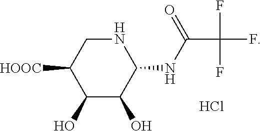 [SMILES: O=C(O)[C@H]1CN[C@H](NC(=O)C(F)(F)F)[C@@H](O)[C@H]1O ; Cl]

20.-22. (canceled)

23. The method of claim 18, wherein the tissue is contacted with the heparanase inhibitor compound while the tissue is in a donor of the tissue or the tissue is contacted with the heparanase inhibitor compound ex vivo or in vitro.

24. (canceled)

25. The method of claim 18, wherein the tissue is contacted with the heparanase inhibitor compound after implantation in a recipient of the tissue.

26. The method of claim 18, wherein the tissue is in the form of an organ, and is optionally-perfused with a perfusate comprising the heparanase inhibitor compound.

27. (canceled)

28. The method of any claim 18, wherein the tissue is contacted with a solution of the heparanase inhibitor compound in which the concentration of the heparanase inhibitor compound ranges from 10 nM to 5 mM.

29. (canceled)

30. The method of claim 28, wherein the tissue is contacted with the heparanase inhibitor compound in a single bolus, or in multiple doses, intermittently, or continuously over a time period ranging from 10 minutes, 20 minutes, 30 minutes, 45 minutes, 1 hour, 2 hours, 3 hours, 4 hours, 5 hours, 6 hours, 12 hours, or 24 hours, or any increment therebetween.

31. The method of claim 18, wherein the tissue comprises lung tissue.

32. The method of claim 18, wherein the heparanase inhibitor compound is heparastatin, which is contacted with the tissue at a concentration of at least 10 nM, at least 50 nM, at least 100 nM, at least 500 nM, at least 1 μM, at least 10 μM, at least 25 μM, or at least 50 μM, and, optionally, no greater than 5 mM, or wherein the heparanase inhibitor compound is contacted with the tissue at a therapeutic equivalent concentration to a concentration of SF-4 (heparastatin) of at least 10 nM, at least 50 nM, at least 100 nM, at least 500 nM, at least 1 μM, at least 10 μM, at least 25 μM, or at least 50 μM, and, optionally, no greater than 5 nM.

33. (canceled)
